(19) United States
(12) Patent Application Publication
(10) Pub. No.: US 2026/0311852 A1
(43) Pub. Date: Oct. 8, 2026

(54) Methods and Apparatus for Benchmarking Compliance

(71) Applicant: ResMed Digital Health Inc., San Diego, CA (US)

(72) Inventor: Shweta PAI, Halifax, Nova Scotia (CA)

(73) Assignee: ResMed Digital Health Inc., San Diego, CA (US)

(21) Appl. No.: 19/481,010

(22) PCT Filed: May 6, 2024
(86) PCT No.: PCT/US2024/028010

§ 371 Date: Nov. 3, 2025

Related U.S. Application Data
(60) Provisional application No. 63/500,083, filed on May 4, 2023.

(51) Int. Cl. G16H 40/20; G16H 40/67

(52) U.S. Cl. CPC G16H 40/20; G16H 40/67

(57) ABSTRACT

A system (100) monitors performance associated with home medical equipment. The system may include a non-transitory computer-readable medium that stores information for each of a plurality of patients. At least one processor may be configured to receive, from a plurality of HME devices, device usage of the plurality of patients. The processor may determine whether at least one of the plurality of patients is a compliant patient by comparing the device usage of the at least one patient to a device usage requirement. The processor may receive a filter criterion, from a user, the filter criterion concerning the information. A performance indicator may be output to the user, based on a number of compliant patients whose information meets the filter criterion.

### 1 CROSS REFERENCE TO RELATED APPLICATIONS

This application claims the benefit of U.S. Provisional Patent Application No. 63/500,083, filed May 4, 2023, the entire content of which is incorporated herein by reference.

### 2 BACKGROUND OF THE TECHNOLOGY

### 2.1 Field of the Technology

The present technology relates to providing data and analytics related to compliance of therapy requirements. In particular, the present technology relates to monitoring compliance performance associated with home medical equipment (HME), such as a respiratory therapy device.

### 2.2 Description of the Related Art

Many people require the use of therapy devices, such as on a daily basis. For example, it is believed that more than 8 million positive airway pressure (e.g., CPAP) devices are sold annually in the United States, with another 2.5 million globally. When using an HME device, a patient may need to continuously comply with a certain compliance, therapy or device usage requirement or standard in order to receive the maximum benefit provided by the HME device.

Patient compliance with compliance requirements may benefit a HME provider who provides the HME device to the patient(s). Thus, thousands of HME providers, which may each manage hundreds of patients, may have systems and/or personnel to assist, such as with instruction and coaching, the patient on how to use the HME device. The HME provider may be eligible to receive a reimbursement from the patient's insurer (e.g., insurance company) when the patient uses the HME device pursuant to his/her compliance requirement. An example compliance requirement may be a standard provided by Centers for Medicare & Medicaid Services (CMS) or a standard provided by the Medica Medicare Medicaid, which, for example, may require the patient to use a respiratory therapy type HME device for at least four hours for 21 out of 30 days in a 90-day window period, in order for the HME provider to receive the reimbursement. Furthermore, a patient who continuously meets the compliance requirement may be a candidate for frequent resupply of HME device accessories such as masks and tubes.

As a result, the HME providers are incentivized to have systems and/or clinicians for motivating the patients on using the HME device to meet the compliance requirement. Some HME providers have systems/clinicians in different locations that provide coaching services to the patients.

Existing technologies merely assess compliance of individual patients, but do not provide broader benchmark assessments, such as for HME providers with respect to their performance, such as in relation to coaching and/or assisting their patients to remain compliant. Typical prior technologies do not provide for practical access to, or analysis of such data, let alone sufficient data, nor can such data be readily evaluated or understood to provide such benchmark assessments since such information is generally widely dispersed and not well suited to tracking and evaluation, such as when vast amounts of data is necessary for benchmarking assessments.

For each HME provider, there is a need to understand performance of its systems/clinicians and locations, as well as performance benchmarks in the industry, which may help the HME provider make improved decisions with respect to taking suitable actions to improve its performance, which in turn may improve patient health as a collective.

### 3 BRIEF SUMMARY OF THE TECHNOLOGY

The present technology is directed towards systems for monitoring performance of HME providers, clinicians, physicians or insurers, measured by their patients' compliance success when using HME devices.

A first aspect of the present technology relates to a system for monitoring performance associated with HME.

Some implementations of the present technology may include a system for monitoring performance associated with home medical equipment (HME). The system may include a non-transitory computer-readable medium that stores information for each of a plurality of patients, which may include, for each patient, any one or more of: (a) at least one coach identifier identifying at least one coach who coaches the patient, and (b) a geographic location where the patient receives coaching from the at least one coach. The system may include one or more processors. The one or more processors may be configured to access device usage data of the plurality of patients that may be repeatedly received from a plurality of HME devices. The one or more processors may be configured to determine whether each of the plurality of patients is a compliant patient by comparing the device usage data to one or more device usage requirements. The one or more processors may be configured to receive a filter criterion or criteria, from a user. The filter criterion may include at least one selected coach identifier and/or at least one selected geographic location. The one or more processors may be configured to generate a performance indicator, for output to the user, based on a number of compliant patients whose information meets the filter criterion.

In some implementations, a device usage data requirement of the one or more device usage requirements may indicate a predetermined number of device operation hours. The at least one coach of each patient may be a clinician and/or a physician. The performance indicator may include a ratio between the number of compliant patients and a total number of the plurality of patients. The number of compliant patients may be a number of compliant patients, coached by at least one coach identified by the at least one selected coach identifier. The performance indicator may be configured to indicate a progression of the number of compliant patients, coached by the at least one coach identified by the at least one selected coach identifier, over time. The performance indicator may be configured to indicate a comparison between the number of compliant patients, coached by the at least one coach identified by the at least one selected coach identifier and an acceptable threshold. The acceptable threshold may be an average number of compliant patients per coach. The one or more processors may be configured to generate a message when the number of compliant patients, coached by the at least one coach identified by the at least one selected coach identifier, falls below the acceptable threshold. The one or more processors may be configured to send the generated message to the at least one coach identified by the at least one selected coach identifier.

In some implementations, the at least one coach identified by the at least one selected coach identifier may include at least one clinician and/or at least one physician. The performance indicator may include an average number of patients per coach. The number of compliant patients may be a number of compliant patients, coached at the at least one selected geographic location. The number of compliant patients may be a number of compliant patients, coached by at least one coach identified by the at least one selected coach identifier at the at least one selected geographic location. The at least one selected geographic location may include at least two selected geographic locations. The performance indicator may include a ratio between a number of compliant patients and a total number of patients coached at one of the at least two selected geographic locations; and a ratio between a number of compliant patients and a total number of patients coached at another one of the at least two selected geographic locations. The plurality of HME devices may be provided to the plurality of patients by an HME provider.

Some implementations of the present technology may include a system for monitoring performance associated with home medical equipment (HME). The system may include a computer-readable medium that stores information for each of a plurality of patients, including, for each patient, any one or more of: (a) an HME provider identifier identifying one of a plurality of HME providers, wherein the one may be associated with the patient, (b) a physician identifier identifying a physician who coaches the patient on using a HME device; (c) a geographic location where the patient receives coaching for using the HME device; and (d) an insurer identifier identifying an insurer associated with the patient. The system may include one or more processors. The one or more processors may be configured to access device usage data of the plurality of patients that may be repeatedly received from a plurality of HME devices, where the HME devices may be provided to the patients by a plurality of HME providers. The one or more processors may be configured to determine whether each of the plurality of patients may be a compliant patient by comparing the device usage data to one or more device usage requirements. The one or more processors may be configured to receive a filter criterion or criteria, from a user. The filter criterion may include any one or more of the following: at least one selected HME provider identifier, at least one selected physician identifier, at least one selected geographic location, and at least one selected insurer identifier. The one or more processors may be configured to generate a performance indicator, for output to the user, based on a number of compliant patients whose information meets the filter criterion.

In some implementations, each of the plurality of HME providers may have a plurality of patients. The at least one selected HME provider identifier may include at least two selected HME provider identifiers, and the performance indicator may include: a ratio between a number of compliant patients and a total number of patients associated with one of the at least two selected HME provider identifiers; and a ratio between a number of compliant patients and a total number of patients associated with another one of the at least two selected HME provider identifiers. The number of compliant patients may be a number of compliant patients associated with at least one HME provider identified by the at least one selected HME provider identifier. The performance indicator may be configured to indicate a comparison between (1) a ratio between the number of compliant patients and a total number of patients associated with the at least one HME provider identified by the at least one selected HME provider identifier and (2) an average ratio between a number of compliant patients and a total number of patients per HME provider of a patient volume similar to the at least one HME provider identified by the at least one selected HME provider identifier.

In some implementations, the one or more processors may be configured to generate a message when the ratio associated with the at least one HME provider identified by the at least one selected HME provider identifier falls below the average ratio. The one or more processors may be configured to send the message to the at least one HME provider identified by the at least one selected HME provider identifier. The number of compliant patients may be a number of compliant patients associated with at least one HME provider identified by the at least one selected HME provider identifier, and coached at the at least one selected geographic location. The performance indicator may be configured to indicate a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider. The performance indicator may be configured to indicate a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider of a patient volume similar to the at least one HME provider identified by the at least one selected HME provider identifier. The number of compliant patients may be a number of compliant patients, coached by at least one physician identified by the at least one selected physician identifier. The performance indicator may include a ratio between a number of compliant patients and a total number of patients associated with at least one HME provider identified by the at least one HME provider identifier and coached by at least one physician identified by the at least one selected physician identifier. The performance indicator may be configured to indicate a comparison between (1) the ratio and (2) an average ratio between a number of compliant patients and a total number of patients coached by at least one physician identified by the at least one selected physician identifier across the plurality of HME providers. The performance indicator may include a ratio between a number of compliant patients and a total number of patients associated with at least one insurer identified by the at least one insurer identifier. The performance indicator may be configured to indicate a comparison between (1) the ratio and (2) an average ratio between a number of compliant patients and a total number of patients per insurer.

Some implementations of the present technology may include a method for monitoring performance associated with home medical equipment (HME). The method may include storing, by a computer-readable medium, information for each of a plurality of patients, wherein the information for each patient may include at least one of (or at least one or more of): (a) at least one coach identifier identifying at least one coach who coaches the patient, and (b) a geographic location where the patient receives coaching from the at least one coach. The method may include accessing, by one or more processors, device usage data of the plurality of patients that may be received from a plurality of HME devices. The method may include determining, by the one or more processors, whether each of the plurality of patients is a compliant patient by comparing the device usage data to one or more device usage requirements. The method may include receiving, by the one or more processors, a filter criterion (or criteria), from a user, the filter criterion including at least one selected coach identifier and/or at least one selected geographic location. The method may include generating, by the one or more processors, a performance indicator, for output to the user, based on a number of compliant patients whose information meets the filter criterion.

In some implementations, the device usage requirement may indicate a predetermined number of device operation hours. The at least one coach of each patient may include a clinician and/or a physician. The performance indicator may include a ratio between the number of compliant patients and a total number of the plurality of patients. The number of compliant patients may be a number of compliant patients, coached by at least one coach identified by the at least one selected coach identifier. The performance indicator may be configured to indicate a progression of the number of compliant patients, coached by the at least one coach identified by the at least one selected coach identifier, over time. The performance indicator may be configured to indicate a comparison between the number of compliant patients, coached by the at least one coach identified by the at least one selected coach identifier and an acceptable threshold. The acceptable threshold may be an average number of compliant patients per coach.

In some implementations, the method may further include generating, by the one or more processors, a message when the number of compliant patients, coached by the at least one coach identified by the at least one selected coach identifier, falls below the acceptable threshold. The method may further include sending, by the one or more processors, the generated message to the at least one coach identified by the at least one selected coach identifier. The at least one coach identified by the at least one selected coach identifier may include at least one clinician and/or at least one physician. The performance indicator may include an average number of patients per coach. The number of compliant patients may be a number of compliant patients, coached at the at least one selected geographic location. The number of compliant patients may be a number of compliant patients, coached by at least one coach identified by the at least one selected coach identifier at the at least one selected geographic location. The at least one selected geographic location may include at least two selected geographic locations, and the performance indicator may include: a ratio between a number of compliant patients and a total number of patients coached at one of the at least two selected geographic locations; and a ratio between a number of compliant patients and a total number of patients coached at another one of the at least two selected geographic locations. The plurality of HME devices may be provided to the plurality of patients by an HME provider.

Some implementations of the present technology may include a method for monitoring performance associated with home medical equipment (HME). The method may include storing, by a non-transitory computer-readable medium, information for each of a plurality of patients, wherein the information for each patient includes at least one (or more) of: (1) an HME provider identifier identifying one of a plurality of HME providers, wherein the one may be associated with the patient, (2) a physician identifier identifying a physician who coaches the patient on using a HME device; (3) a geographic location where the patient receives coaching for using the HME device; and (4) an insurer identifier identifying an insurer associated with the patient. The method may include accessing, by one or more processors, device usage data of the plurality of patients that may be received from a plurality of HME devices, the HME devices being provided to the patients by a plurality of HME providers. The method may include determining, by the one or more processors, whether each of the plurality of patients may be a compliant patient by comparing the device usage data to one or more device usage requirements. The method may include receiving, by the one or more processors, a filter criterion (or criteria), from a user, the filter criterion may include any one or more of the following: at least one selected HME provider identifier, at least one selected physician identifier, at least one selected geographic location, and at least one selected insurer identifier. The method may include generating, by the one or more processors, a performance indicator, for output to the user, based on a number of compliant patients whose information meets the filter criterion.

In some implementations, the plurality of HME providers are of a plurality of patient volumes. The at least one selected HME provider identifier may include at least two selected HME provider identifiers, and the performance indicator may include: a ratio between a number of compliant patients and a total number of patients associated with one of the at least two selected HME provider identifiers; and a ratio between a number of compliant patients and a total number of patients associated with another one of the at least two selected HME provider identifiers. The number of compliant patients may be a number of compliant patients associated with at least one HME provider identified by the at least one selected HME provider identifier. The performance indicator may be configured to indicate a comparison between (1) a ratio between the number of compliant patients and a total number of patients associated with the at least one HME provider identified by the at least one selected HME provider identifier and (2) an average ratio between a number of compliant patients and a total number of patients per HME provider of a patient volume similar to the at least one HME provider identified by the at least one selected HME provider identifier. The method may further include generating, by the one or more processors, a message when the ratio associated with the at least one HME provider identified by the at least one selected HME provider identifier falls below the average ratio. The method may further include sending, by the one or more processors, such as via a network by use of a communications device, the message to the at least one HME provider identified by the at least one selected HME provider identifier.

In some implementations, the number of compliant patients may be a number of compliant patients associated with at least one HME provider identified by the at least one selected HME provider identifier, and coached at the at least one selected geographic location. The performance indicator may be configured to indicate a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider. The performance indicator may be configured to indicate a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider of a patient volume similar to the at least one HME provider identified by the at least one selected HME provider identifier. The number of compliant patients may be a number of compliant patients, coached by at least one physician identified by the at least one selected physician identifier. The performance indicator may include a ratio between a number of compliant patients and a total number of patients associated with at least one HME provider identified by the at least one HME provider identifier and coached by at least one physician identified by the at least one selected physician identifier. The performance indicator may be configured to indicate a comparison between (1) the ratio and (2) an average ratio between a number of compliant patients and a total number of patients coached by at least one physician identified by the at least one selected physician identifier across the plurality of HME providers. The performance indicator may include a ratio between a number of compliant patients and a total number of patients associated with at least one insurer identified by the at least one insurer identifier. The performance indicator may be configured to indicate a comparison between (1) the ratio and (2) an average ratio between a number of compliant patients and a total number of patients per insurer.

Some implementations of the present technology may include a system for monitoring performance associated with home medical equipment (HME). The system may include a computer-readable medium configured to store information for each of a plurality of patients. The system may include one or more processors. The one or more processors may be configured to access device usage data of the plurality of patients that may be repeatedly received from a plurality of HME devices. The one or more processors may be configured to determine whether each of the plurality of patients may be a compliant patient by comparing the device usage data to one or more device usage requirements. The one or more processors may be configured to receive a filter criterion (or criteria), from a user, the filter criterion concerning the information. The one or more processors may be configured to generate a performance indicator, for output to the user, based on a number of compliant patients whose information meets the filter criterion.

In some implementations, the information for each patient includes at least one (or more) of: (1) at least one coach identifier identifying at least one coach who coaches the patient, and (2) a geographic location where the patient receives coaching from the at least one coach. The filter criterion may include at least one selected coach identifier, at least one selected coach type, at least one therapy device type, and/or at least one selected geographic location. The HME devices may be provided to the patients by a plurality of HME providers. The information for each patient may include at least one (or more) of: (1) an HME provider identifier identifying one of the plurality of HME providers associated with the patient, (2) a physician identifier identifying a physician who coaches the patient on using the HME device; (3) a geographic location where the patient receives coaching for using the HME device; (4) an insurer identifier identifying an insurer associated with the patient, and (5) a therapy device type. The filter criterion may include any one or more of the following: at least one selected HME provider identifier, at least one selected physician identifier, at least one selected geographic location, and at least one selected insurer identifier. The performance indicator may be configured to indicate an average number of compliant patients coached by an HME provider on a monthly basis.

Some implementations of the present technology may include a method for monitoring performance associated with home medical equipment (HME). The method may include storing, by a computer-readable medium, information for each of a plurality of patients, such as in one or more databases. The method may include accessing, by one or more processors, device usage data of the plurality of patient that may be repeatedly received from a plurality of HME devices. The method may include determining, by the one or more processors, whether each of the plurality of patients may be a compliant patient by comparing the device usage data to one or more device usage requirements. The method may include receiving, by the one or more processors, a filter criterion (or criteria), from a user, the filter criterion concerning the information. The method may include generating, by the one or more processors, a performance indicator, for output to the user, based on a number of compliant patients whose information meets the filter criterion.

In some implementations, the information for each patient may include at least one of: (1) at least one coach identifier identifying at least one coach who coaches the patient, and (2) a geographic location where the patient receives coaching from the at least one coach. The filter criterion may include at least one selected coach identifier, at least one selected coach type, at least one therapy device type, and/or at least one selected geographic location. The HME devices may be provided to the patients by a plurality of HME providers. The information for each patient may include at least one (or more) of: (1) an HME provider identifier identifying one of the plurality of HME providers associated with the patient, (2) a physician identifier identifying a physician who coaches the patient on using the HME device; (3) a geographic location where the patient receives coaching for using the HME device; (4) an insurer identifier identifying an insurer associated with the patient, and (5) a therapy device type. The filter criterion may include one or more of the following: at least one selected HME provider identifier, at least one selected physician identifier, at least one selected geographic location, and at least one selected insurer identifier. The performance indicator may be configured to indicate an average number of compliant patients coached by an HME provider on a monthly basis.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Monitoring System

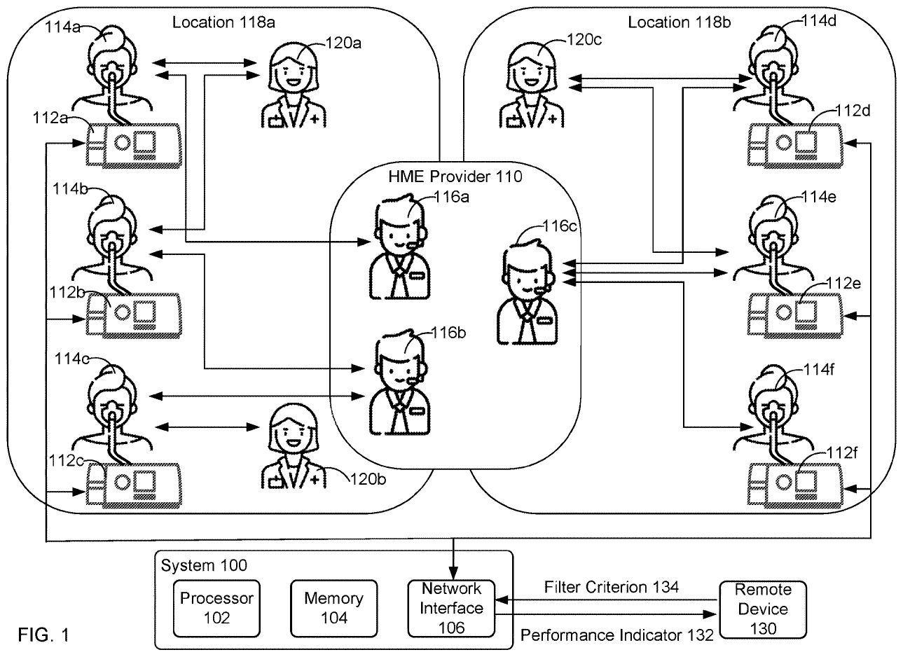

FIG. 1 illustrates an environment in which a system monitors performance associated with HME, according to one aspect of the present disclosure.

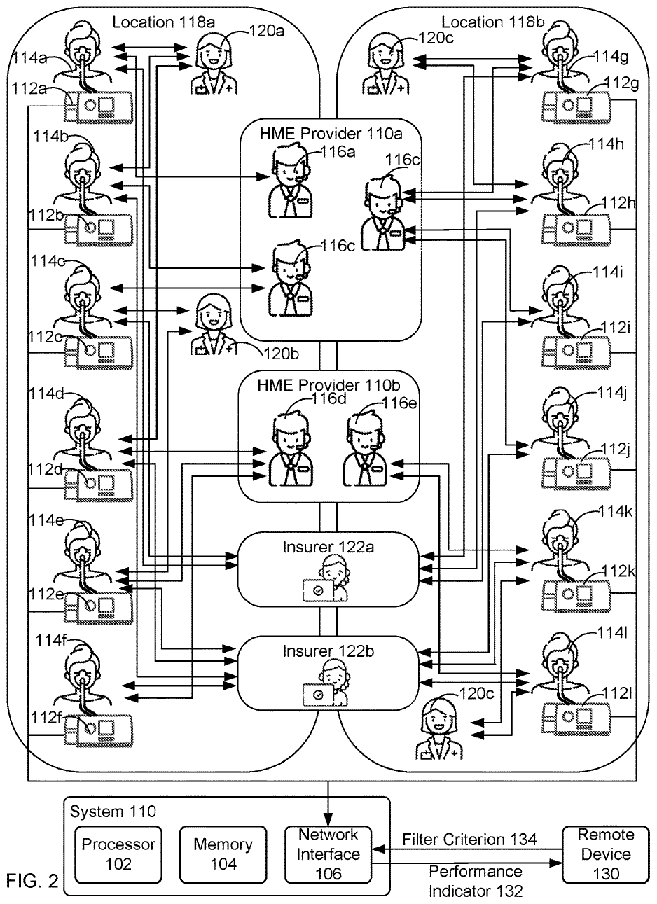

FIG. 2 illustrates another environment in which a system monitors performance associated with HME, according to one aspect of the present disclosure.

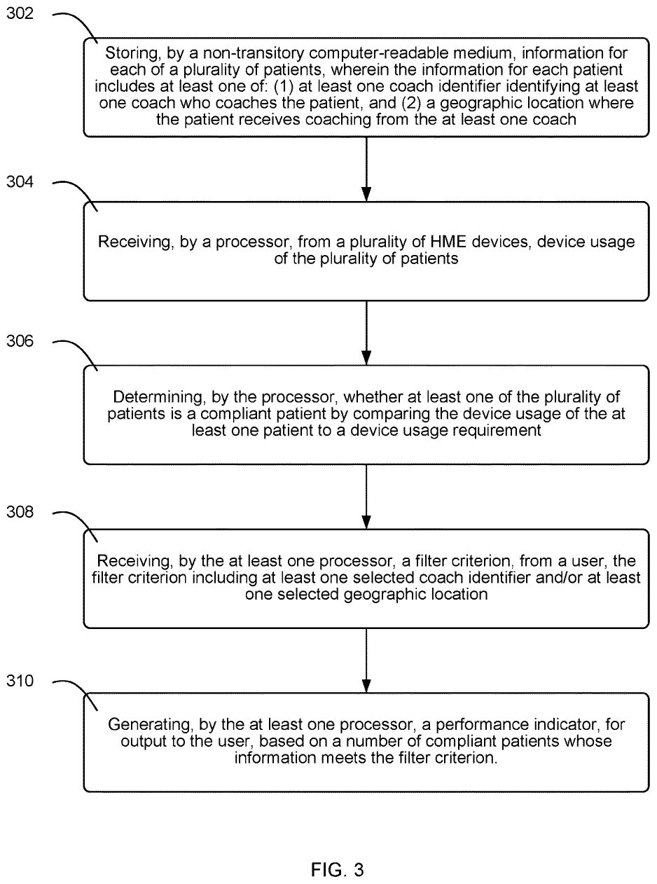

FIG. 3 illustrates an example flow chart of a method for monitoring performance associated with HME, according to one aspect of the present technology.

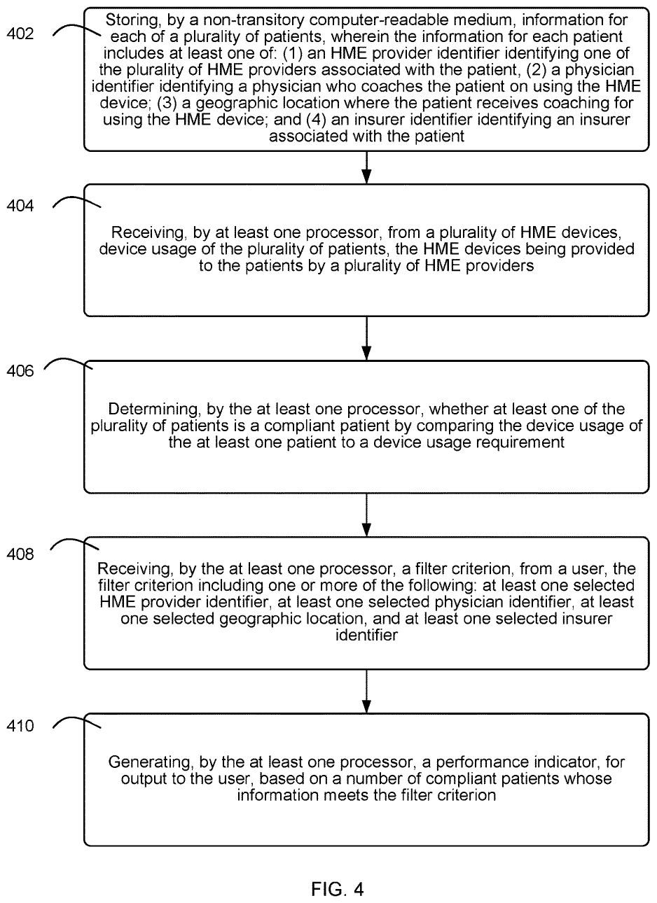

FIG. 4 illustrates another example flow chart of a method for monitoring performance associated with HME, according to one aspect of the present technology.

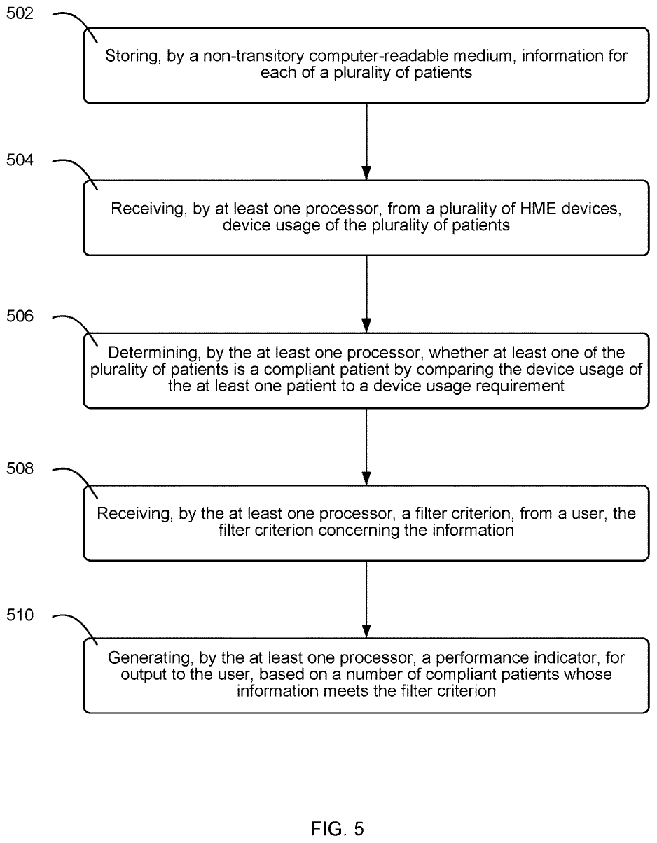

FIG. 5 illustrates yet another example flow chart of a method for monitoring performance associated with HME, according to one aspect of the present technology.

### 4.2 Respiratory Therapy Systems

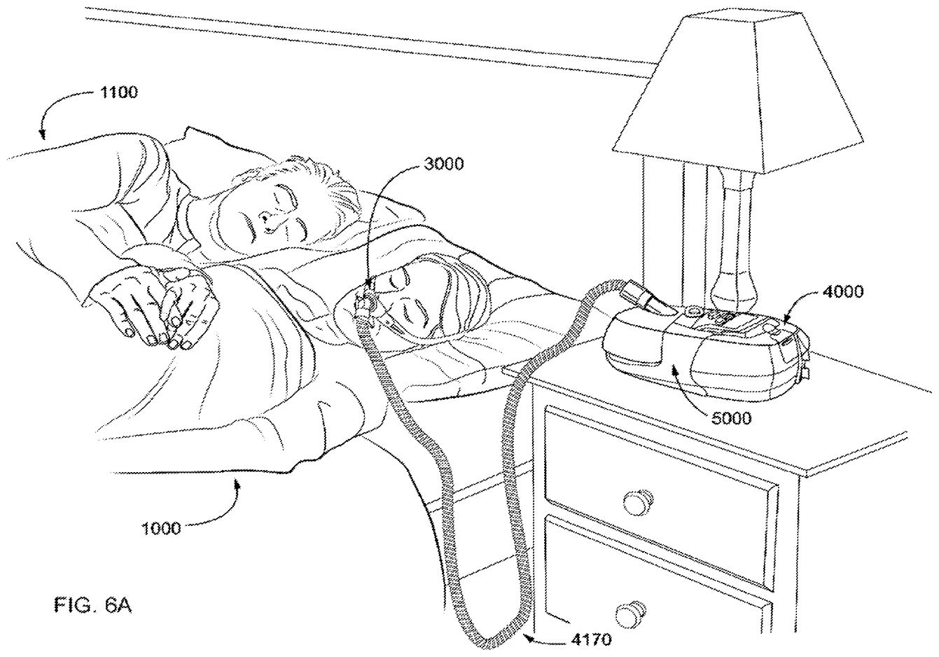

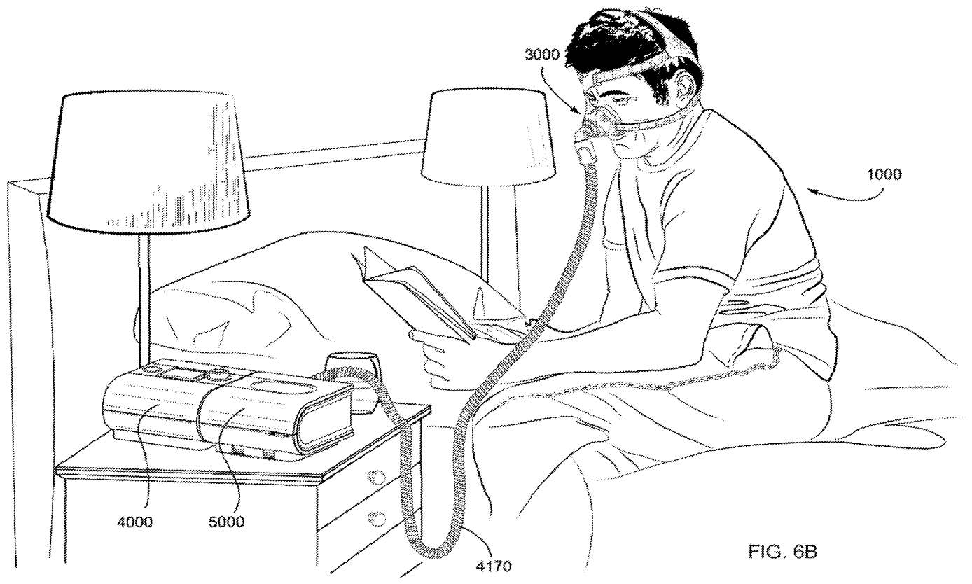

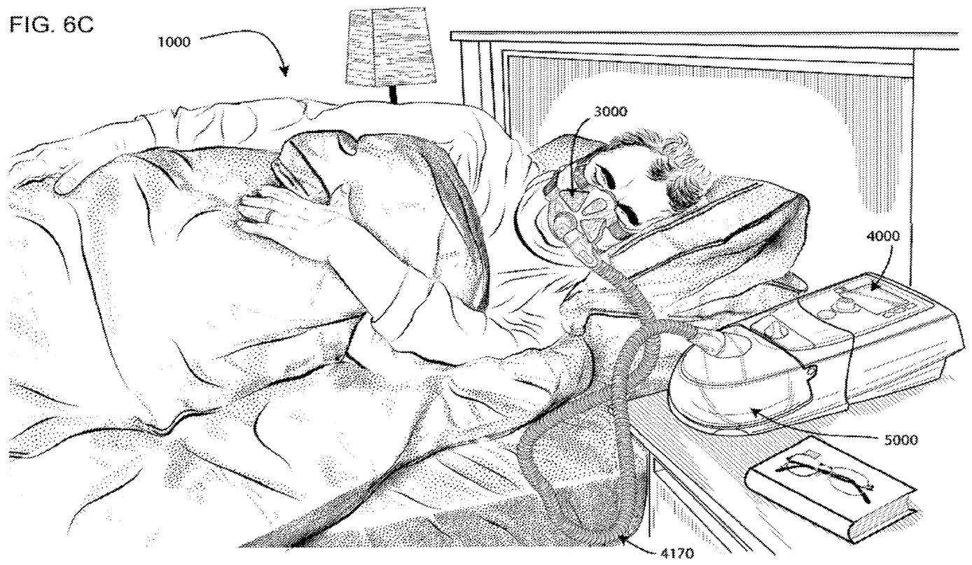

FIG. 6A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 6B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 6C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.3 RPT Device

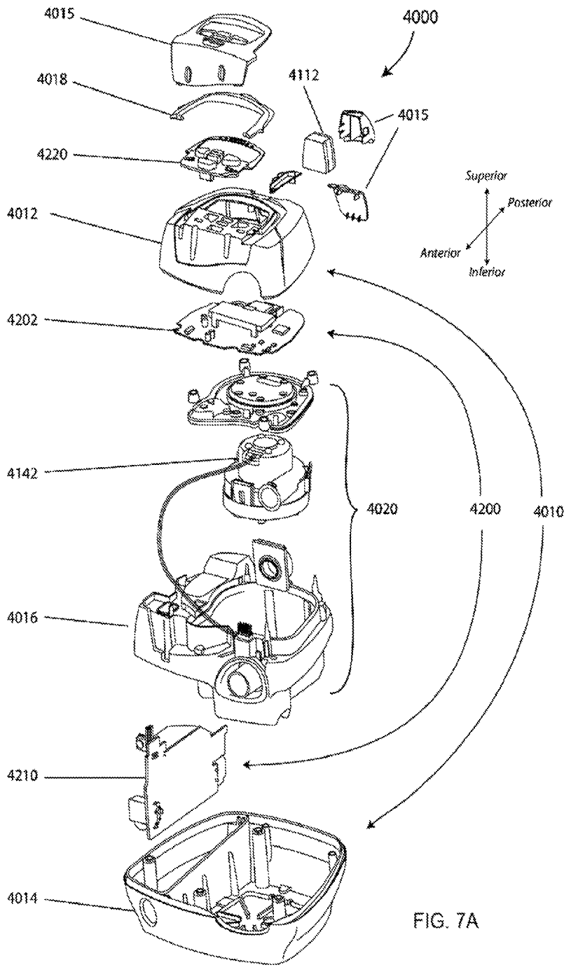

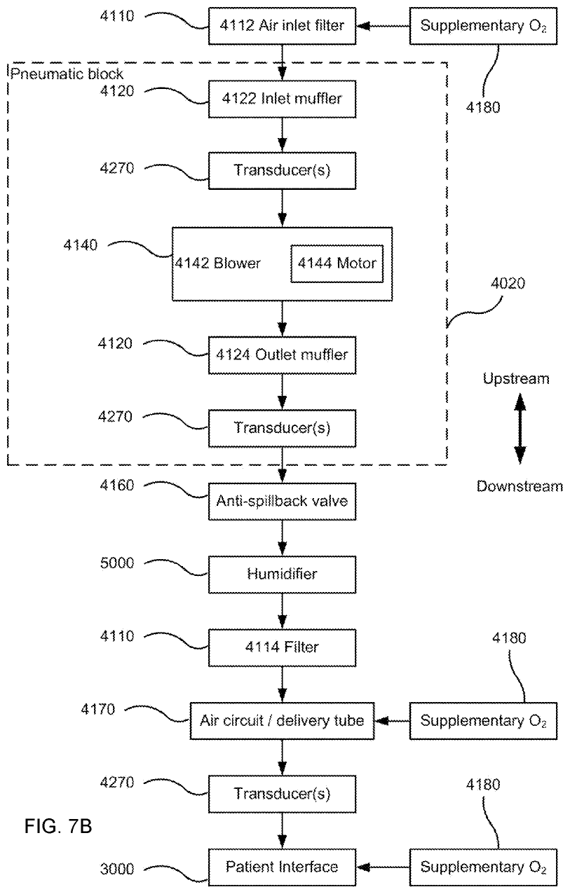

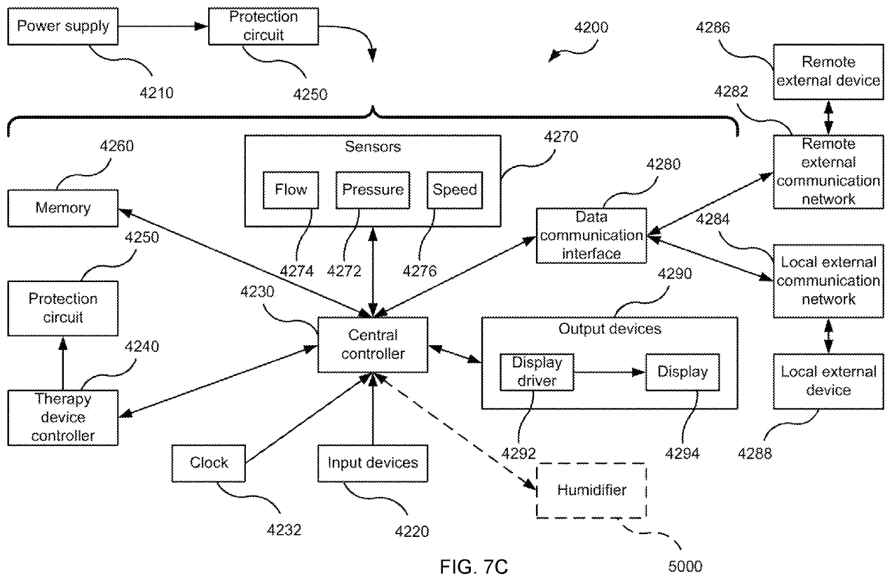

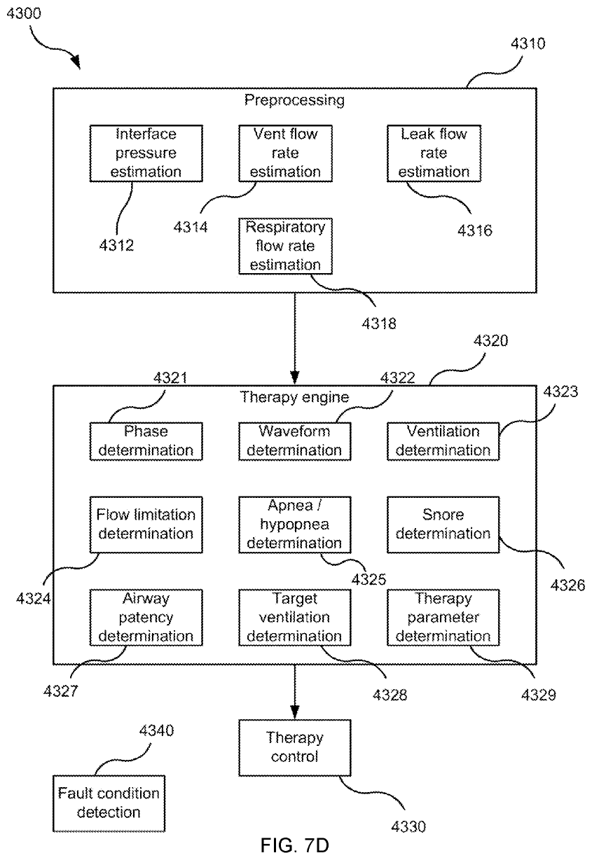

FIG. 7A shows an RPT device in accordance with one form of the present technology.

FIG. 7B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 7C is a schematic diagram of the electrical components of an RPT device in accordance with one form of the present technology.

FIG. 7D is a schematic diagram of the algorithms implemented in an RPT device in accordance with one form of the present technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Monitoring System

FIG. 1 illustrates a technology environment in which an example system 100 monitors performance associated with HME. As shown in FIG. 1, an HME provider 110 may provide one or more HME devices 112a-f to one or more patients 114a-f, and hire one or more clinicians 116a-c to coach the patients 114a-f on how to use the HME devices 112a-f to meet a compliance, adherence, or therapy requirement. Each HME device may be a respiratory therapy (RPT) device, details of an example of which are discussed in section 4.2. The compliance requirement may be a device usage standard, which typically requires a predetermined number of device operation hours or therapy hours with the device. The predetermined number of device hours may represent an expected amount of time that a patient should use an HME device for within a predetermined period of time. In one example, the device usage requirement may require the patient to use the HME device for at least four hours for 21 out of 30 days in a 90-day window period. For each patient who meets the device usage requirement, the HME provider may get a reimbursement from the patient's insurer (e.g., insurance company).

The HME provider 110 may provide coaching services to the patients at different geographic locations 118a and 118b. The geographic location 118a or 118b may refer to a country, state, province or city, among other possibilities. It one example, the HME provider 100 may coach patients 114a-f in Florida, Texas, and North Carolina. The HME provider may provide different coaches for a particular patient such that different coaches may have different responsibilities regarding a patient's use of a therapy device. For example, a clinician or other such coach may be characterized by type. For example, a clinician coach may be a setup coach type so as to designate the coach as a person involved with initial training of a patient with regard to use and setup of a therapy device. By way of further example, a clinician coach may be a resupply coach type so as to designate the coach as a person involved with arranging for replacement of therapy products such as consumables (e.g., a mask or filter) to the patient with regard to use of a therapy device or a newer replacement therapy device. Similarly, in some implementations a clinician coach may be a contact coach type so as to designate the coach as a person involved with communication with the patient and monitoring of the patient's ongoing therapy for promoting the patient's continued use of the therapy device such as when use time declines.

The HME provider 110 may partner with one or more physicians 120a-c. Each patient may receive coaching not only from a clinician of the HME provider 110, but also optionally from a physician. The relationship between a patient and his/her clinician and/or physician may impact the patient's compliance to the compliance requirement. If a provider and/or clinician/physician has a better relationship with the patient, the patient may be more likely to meet the compliance requirement. When a patient receives coaching from both the clinician and the physician, the patient may have an increased chance to comply with the compliance requirement.

The system 100 may monitor performance of one or more HME providers 110, one or more clinicians 112a, 112b and 112c, one or more locations 118a and 118b where the clinicians provide that aforementioned coaching, or types of coaching, to the patients, one or more physicians 120a, 120b and 120c who also provide coaching to the patients regarding use of the HME devices, and one or more insurers who provide insurance to the patients. Such monitoring may be based on receiving, such as via networked communications, compliance related data from patients' HME devices, which data from each device may then be associated with a particular HME provider (e.g., name and/or location) and/or a particular clinician, and/or a particular physician, and/or a particular insurer, etc. The system 100 may evaluate each performance such as by measuring each performance respectively based on a percentage or a number of patients who comply with one or more compliance requirements. For example, such a performance measure may include a ratio of a total number of compliant patients associated with a particular HME provider to the total number of patients of the HME provider, such as for a particular type of HME device, which ratio may be presented, for example, as a percentage. Similarly, such a performance measure may include a ratio of a total number of compliant patients associated with a particular location of an HME provider to the total number of patients of the location of the HME provider, such as for a particular type of HME device, which ratio may be presented, for example, as a percentage. Still similarly, such a performance measure may include a ratio of a total number of compliant patients associated with a particular clinician to the total number of patients of the clinician, such as for a particular type of HME device, which ratio may be presented, for example, as a percentage. Still similarly, such a performance measure may include a ratio of a total number of compliant patients associated with a particular physician to the total number of patients of the physician, such as for a particular type of HME device, which ratio may be presented, for example, as a percentage. Such a measure(s) may be presented in, for example, a report or output to a display with a graphic user interface, such as in response to a query to the system 100, that may be input or initiated with the graphic user interface, such as on a remote device 130 that accesses the system 100. The measure may be generated as an indicator or a part thereof, such as a graphic indicator presented on some output such as a display, such that the indicator may represent information regarding one or more of the performance measures. Such indicator(s) may be presented as numeric and/or graphic image data.

Thus, the system 100 may include one or more processors 102 (such as one or more servers), memory 104, and a network interface 106. The system 100 may have programming, such as one or more application programming interface(s) (API), that may be integrated with other systems for access to the system 100, such as to generate or communicate with such a graphic user interface and/or respond to one or more of such queries, such as with one or more remote device(s) 130, for generating such reports and outputs.

The processor(s) 102 may be a dedicated electronic circuit or an application-specific integrated circuit. The processor(s) 102 may be formed with discrete electronic components. The processor(s) 102 may include a dedicated motor control integrated circuit. The processor(s) 102 may include one or more processors or microprocessors, configured to execute one or more computer programs stored in the memory 104.

The memory 104 may be a non-transitory computer-readable medium configured to store one or more algorithms and/or methods described herein in the form of computer program instructions and/or information/data described herein. Thus, the memory 104 may include one or more of the following: non-volatile memory, battery powered static random-access memory (RAM), and volatile RAM. The memory 104 may be in the form of electrically erasable programmable read-only memory (EEPROM) or NAND flash and/or any other secondary data storage device(s). Such memory may also include, for example one or more databases, which may be remote or collocated with the processor(s) 102. Thus, such memory or databases may optionally be remotely accessible, for example, via the network interface 106.

Thus, the memory 104 may store mapping relationships of one or more of the following: HME provider(s), clinician(s), patient(s), physician(s), location(s), insurer(s) and HME device(s), each of which may have a unique identifier. For instance, each patient may have a unique patient identifier. Each clinician and physician may have a unique coach identifier. Alternatively, each clinician may have a unique clinician identifier, and each physician may have a unique physician identifier. Each insurer may have a unique insurer identifier. Each HME device may have a unique HME device identifier. For each HME provider, the memory 104 may store mapping relationships indicating which patients, clinicians, physicians and insures are associated with the HME provider. For each patient, the memory may store mapping relationships indicating which HME provider, clinician and insurer serve the patient. If the patient has a physician, the memory may also store a mapping relationship reflecting association between the patient and the physician. For each physician, the memory store mapping relationships indicating which HME provider and patients are associated with the physician. For each insurer, the memory may store mapping relationships indicating which HME providers, patients, and physicians are associated with the insurer. Optionally, the memory may also store coach type designators for each coach or clinician so as to provide a characterization of such coaches by coach type such as any one or more of the aforementioned coach types.

The memory 104 may store information for each patient. In a first example, the memory may store, in addition to aggregating the voluminous usage and/or compliance data for multitudes of therapy sessions for each HME device regarding patient device use and/or compliance, one or more of the following information for each patient: (1) at least one coach identifier identifying at least one coach who coaches the patient, and (2) a geographic location where the patient receives coaching from the at least one coach. The at least one coach of each patient may include a clinician and/or a physician.

In a second example, the memory 104 may store one or more of the following information for each patient: (1) an HME provider identifier identifying one of the plurality of HME providers associated with the patient, (2) a physician identifier identifying a physician who coaches the patient on using the HME device; (3) a geographic location where the patient receives coaching for using the HME device; and (4) an insurer identifier identifying an insurer associated with the patient.

To illustrate the first example, for a system 100 that provides intra-organization performance indicators as shown in FIG. 1, in addition to the usage data and/or compliance data, the memory 104 may store information associated with each patient, including one or more of the following: at least one coach identifier identifying a coach who coaches the patient, a geographic location where the patient receives coaching from, a patient identifier that identifies the patient, an HME device identifier that identifies the HME device used by the patient, and an insurer identifier that identifies an insurer who provides insurance to the patient. A patient may have one or two coaches, such as a clinician and/or a physician. The coach identifier may include a clinician identifier and/or a physician identifier. In addition, the memory 104 may store information associated with each clinician, including one or more of the following: a coach identifier or a clinician identifier that identifies each clinician, and one or more geographic locations where each clinician provides coaching, and optionally a coach type designator(s). The memory 104 may also store information associated with each physician, including one or more of the following: a coach identifier or a physician identifier that identifies the physician, one or more geographic locations where the physician provides coaching, and optionally a coach type designator(s), and one or more insurer identifiers that identify insurer(s) who partner with the physician.

To illustrate the second example, for a system 100 that provides industry performance indicators as shown in FIG. 2, in addition to the usage data and/or compliance data, the memory 104 may store the following additional information, reflecting patient, clinician and physician relationships with respect to HME providers. For each HME provider, the memory 104 may store an HME provider identifier, one or more associated clinician identifiers identifying clinician(s) who work for the HME provider, one or more associated physician identifiers identifying physician(s) who work for the HME provider, and one or more associated patient identifiers identifying patient(s) who receive coaching from the HME provider, and one or more associated insurer identifiers identifying insurer(s) who partner with the HME provider. In addition, with respect to each patient, the memory 104 may store an HME provider identifier that identifies the HME provider associated with the patient. Similarly, with respect to each clinician and each physician, the memory 104 may store an HME provider identifier that identifies the HME provider associated with the clinician and the physician.

As previously noted, the network interface 106 may repeatedly communicate with one or more HME devices 112a-f and one or more remote devices 130 by using a wired and/or wireless communication interface(s). The wired communication interface 1008 may include a wired protocol, to allow, for example, communication via a network, such as an internet or the Internet via Ethernet or optical fibre. The wireless communication interface may include one or more transceivers using wireless protocols such as infrared protocol, cellular, Bluetooth, WIFI, Bluetooth LE, and Bluetooth BLE (5.0) with GATT profile, among other possibilities.

In one example, each HME device may repeatedly report device usage and/or compliance information, along with the HME device identifier, to the processor(s) 102 via the network interface 106 such as for storing within the memory 104 for analysis. Based on such information, the processor(s) 102 may identify patients who meet or adhere to the compliance requirement, such as to by evaluation of usage and/or compliance information and/or by creating compliance information from usage information.

Thus, the processor(s) 102 may repeatedly determine whether each patient is a compliant patient by comparing the device usage of each patient to the device usage requirement, by repeated access to the memory 104. Such compliance information may also be stored in the memory 104, such as in association with the patient, and/or any one or more of the clinician of the patient, the physician of the patient, the HME of the patient, the insurer of the patient, etc.

Thus, the processor(s) 102 may generate one or more performance indicators 132 for output to a user. Such a user may include, but not limited to, clinician(s), decision maker(s) within HME provider(s) 110, such as manager(s) of the clinician(s), physician(s) and insurer(s), among other possibilities. In some implementations, different users at different hierarchical levels within the HME provider 110 may have access to different information. In one example, the system 100 may be accessed by a clinician or his/her manager to view the clinician's performance over a period of time, e.g., over the last few months.

The processor(s) 102 may receive a filter criterion 134 from the user, any one or more of which may be presented as visually selectable options for selection by a user on a graphic user interface (GUI) of the system and/or remote device. The filter criterion 134 may concern information stored in the memory 104. In one example, the filter criterion may include at least one selected coach identifier, at least one coach type, and/or at least one selected geographic location. The at least one selected coach identifier may identify at least one clinician and/or at least one physician and/or coach type. In another example, the filter criterion may include one or more of the following: at least one selected HME provider identifier, at least one selected physician identifier, at least one selected geographic location, and at least one selected insurer identifier. In turn, the processor(s) 102 may generate a performance indicator, for output to the user, based on a number of compliant patients whose information meets the filter criterion. Optionally, such a filter criterion may include, for example, a particular period of time, such as to provide a performance indicator attributable to the particular period (e.g., for a current year, a prior year(s), current month, prior month(s), week, etc.)

In one example, the user may access the system 100 via the remote device 130. The remote device 130 may be a mobile phone, tablet, laptop, personal computer, or other computing or computer device. The remote device 130 may communicate wirelessly with the system 100.

The user may submit the filter criterion 134 from the remote device 130 to the system 100. The filter criterion may include a single filter or an aggregated filter. In one example, a single filter may select a single physician or a single location. In another example, the aggregated filter may select one or more physicians and/or locations.

After receiving the filter criterion 134, the system 100 may generate the performance indicator 132 for output to the user. The system may display the performance indicator 132, such as in the GUI, in an easy way for the user to review through. Such output may be presented to the user within a GUI or other report in any number of ways such as by presenting performance indicator(s) numerically and/or graphically such as with bar and/or line graphs, and multiple such graphs may be presented for comparing a plurality of performance indicators. Optionally, larger bars may represent proportionally larger performances and/or different color bars may be coded to higher and lower performances. In some cases, components of a performance indicator may be graphically displayed such as to proportionally depict the components of an indicator (e.g., each of a total number of patients and a number of compliant patients).

The performance indicator 132 may include an aggregated performance report and/or insight. For example, the performance indicator may provide one or more performance benchmarks to the user, help the user identify any performance decrease, and where, how and when the performance decrease occurs. Various examples of performance benchmarks generated by the system 100 are described below. Thus, the output display may comprise output determined by the system based on a comparison between different ones of a plurality of performance indicators, such as to indicate differences between such performance indicators such as on an HME provider basis (e.g., display of a decrease or increase of a particular HME provider's performance indicators for different periods, and/or a display of differences between the performance indicators of different HME providers for a period of time, etc.). Similarly, such display comparisons may be determined on a clinician basis, a physician basis and/or on a location basis, or any combination thereof. Similarly, other statistical assessments of a plurality of performance indicators may be determined and generated by the system. Moreover, the system may be configured to automatically and repeatedly determine and track such performance indicators and may generate message(s) such as based on one or more comparison(s) between any one or more performance indicators and one or more threshold(s).

By way of example, and as discussed in more detail herein, performance indicators may be presented in a display, such as on a monthly basis, to depict performance of a particular organization (e.g., an HME provider organization) in a graph or graphs such as according to compliance rate (e.g., percentage of patient compliance for the organization) which may be depicted along with a national average compliance rate for a plurality of organizations of a nation. Such a display can permit users to see how the organization performed (as a function of patient compliance) relative to the national average, such as for a set of months. Other useful information may be generated for the display such as showing a percentage of organizations above and/or below the national average for such organizations, which may also include showing a percentage of change such as from a prior period or month. Similarly, other useful information may be generated such as showing a percentage of organizations above and/or below a particular organization's compliance rate. Similarly, performance indicators may be presented in a display, such as on a monthly basis and optionally an overall basis, to depict performances of each of several locations of an organization (e.g., an HME provider organization) in a graph(s) such as according to compliance rate (e.g., percentage of patient compliance for the organization) to permit comparing of the different locations. Similarly, performance indicators may be presented in a display, such as on a monthly basis and optionally an overall basis, to depict performances of each of several clinicians of an organization (e.g., an HME provider organization) in a graph such as according to compliance rate (e.g., percentage of patient compliance for the organization) to permit comparing of the different clinicians. In some implementations, compliance related detail may also be displayed, such as to provide further information regarding performance indications. For example, the total numbers of patients who have been setup with home medical devices, a total numbers of them who are compliant and not compliant may be depicted. In some depictions, timing between when patients are setup to when these patients become compliant may be displayed, such as with line graphs over time.

These and additional examples of such performance determinations and displays/messages may be considered in more detail herein.

### 5.1.1 Example Performance Benchmarks

### 5.1.1.1 Intra-Organizational Performance Benchmarks

In one example, referring to FIG. 1, the system 100 may provide benchmarks for use within a HME provider 110 to identify systemic issues within the HME provider. In this example, users of the system 100 may be limited/filtered to people who work inside the HME provider 110, including for example clinicians and/or managers of clinicians within the HME provider 110.

### 5.1.1.1.1 Clinician Performance

The system 100 may determine performance of one or more clinicians hired by the HME provider 110. In one example, each clinician's performance may depend on a number or a percentage of compliant patients coached by the clinician relative to the total number of patients coached by the clinician. In one example, the greater number of compliant patients that a clinician coaches, the better the clinician's performance.

The system 100 may track each clinician's performance over time. For instance, the system 100 may track each clinician's average performance per month. The performance indicator 132 generated by the system 100 may indicate a progression of the number of compliant patients coached by each clinician over time. When the clinician's performance drops below his/her average performance, the system 100 may generate a message. For example, if a clinician on average coaches about 50 compliant patients a month, the system 100 may generate a message when the clinician's performance drops to 30 compliant patients a month. The message may be sent to the clinician and/or the clinician's manager.

In one example, the system 100 may process a filter criterion that identifies a clinician working within the HME provider. The system 100 may output a performance indicator of the identified clinician. The performance indicator may indicate a comparison between performance of the identified clinician and an acceptable threshold. The acceptable threshold may be an average number of compliant patients per coach as determined by the system 100. The performance indicator may indicate a comparison between the number of compliant patients, coached by the clinician and the acceptable threshold. When the number of compliant patients, coached by the clinician, falls below the acceptable threshold, the processor may generate a message. In one example, the processor may send the generated message to the clinician or the clinician's manager.

Sometimes a clinician may work at multiple locations. When the system 100 receives an aggregated filter identifying a particular clinician at a particular location, the system 100 may generate a performance indicator for that particular clinician at that particular location.

In another example, the system 100 may process a filter criterion that identifies two or more clinicians working for the same HME provider. The system 100 may output a performance comparison between these identified clinicians. Such comparison may help managers or decisionmakers within the HME provider understand deviations in clinicians' performance. If one clinician has a better performance than another clinician, they may share expertise to improve overall performance.

In another example, the system 100 may process a filter criterion that identifies two or more clinicians of the same coaching type who are working for the same HME provider. The system 100 may output a performance comparison between these identified clinicians such as according to clinician type. Such comparison may help managers or decisionmakers within the HME provider understand deviations in clinicians' performance of particular coaching type. If one clinician has a better performance than another clinician of the same coaching type, they may then share expertise to improve overall performance with regard to the particular coaching type.

In one example, the performance indicator may include an average number of patients per coach.

### 5.1.1.1.2 HME Provider Performance

In another example, the system 100 may generate a performance indicator indicating an average number of compliant patients coached by the HME provider on a monthly basis. For example, the system 100 may track performance of each clinician of the HME provider over a period of time, and determine an average number of compliant patients coached by all clinicians of the HME provider per month. Based on how many patients its clinicians can coach, the HME provider may make staffing decisions or adjust workloads.

In another example, the system 100 may output a performance indicator 132 indicating a ratio between a total number of compliant patients and a total number of patients served by the HME provider 110. For instance, the performance indicator 132 may indicate that the HME provider has about 60% compliant patients, meaning that 60% of overall patients served by the HME provider are compliant to the compliance requirement.

In another example, the system 100 may output a performance indicator 132 indicating a ratio between a total number of compliant patients and a total number of patients served by the HME provider 110 with regard to a particular physician. For instance, the performance indicator 132 may indicate that the HME provider has about 60% compliant patients when paired with a particular physician, meaning that 60% of overall patients served by the HME provider with a particular physician are compliant to the compliance requirement.

Moreover, the system may output different performance indicators for the HME according to further characterizations. For example, the system may generate the HME provider performance indicator according to device type and/or patient demographic information (e.g., age, sex, etc.), to provide further characterization of the performance indicator for the HME. For example, the system may generate as output a performance indicator 132 indicating a ratio between a total number of compliant patients who use a particular device and/or are of a particular age, and a total number of patients served by the HME provider 110 who use a particular device and/or are of a particular age.

### 5.1.1.1.3 Location Performance

The system 100 may benchmark location performance. The system 100 may compare performance of different geographic locations where clinicians of the HME provider coach patients. In one example, each location's performance may depend on a number or a percentage of compliant patients coached at that location relative to the total number of patients coached at that location. In one example, the greater the number of compliant patients in a location, the better the performance of that location.

In one example, the system 100 may process a filter criterion 134 that identifies one location. The system 100 may output a performance indicator indicating a number of compliant patients coached at the identified location, which may concern multiple clinicians/physicians.

In another example, the system 100 may process a filter criterion 134 that identifies two locations, such as Florida and Texas. The system 100 may output a performance comparison between these locations. For example, the system 100 may output a performance indicator including: a ratio between a number of compliant patients and a total number of patients coached at a first location, and a ratio between a number of compliant patients and a total number of patients coached at a second location. If one location has a better performance than the other, they may share expertise to improve overall performance.

In yet another example, the system 100 may process a filter criterion 134 that identifies a clinician at a particular location. The system 100 may output a performance indicator indicating a number of compliant patients coached by the identified clinician at the particular location.

### 5.1.1.1.4 Physician Performance

The system 100 may benchmark physician performance. In one example, each physician's performance may depend on a number or a percentage of compliant patients coached by the physician relative to the total number of patients coached by the patient. Physician performance may correlate to the physician's communication efficiency with a patient.

In one example, the system 100 may process a filter criterion 134 that identifies two or more physicians. The system 100 may compare performance or compliance results of these physicians.

In another example, the system 100 may process a filter criterion 134 searching for a top physician (e.g., a physician who provides the best coaching service to patients) in a particular location. The system 100 may output information such as which physician has the highest performance in the particular location.

### 5.1.1.2 Industry Performance Benchmarks

The benchmarks generated by the system 110 may not be limited to a single HME provider. In one example, with reference to FIG. 2, the system 110 may generate one or more benchmarks for the HME industry, which might optionally be further limited according to any of type of HME device (e.g., CPAP devices or ventilator devices) or with collections of types of HME device (e.g., CPAP devices and ventilator devices) or coaching type. For illustrative purposes, FIG. 2 shows HME providers 110a and 110b which staff clinicians 116a-e to provide coaching services to patients 114a-l regarding HME devices 112a-l in locations 118a and 118b. Some patients may work with insurer 122a, while other patients may work with insurer 122b. Here, the HME devices are provided to the patients by the plurality of HME providers 110a and 110b. In this example, users of the system 110 may include decisionmakers of the HME providers, physicians, and insurers, among other possibilities.

As such, the system 100 may benchmark HME providers of similar patient volumes or at same geographic locations, so as to provide a competitive lens into the HME industry based on compliance performance. Various benchmark examples are provided below.

For example, the system 100 may process a filter criterion that identifies a clinician such as of a particular coaching type, and compares that to clinicians of same coaching type who are working for the other HME providers. For example, the system 100 may output a performance indicator comparison between the identified clinician according to any of percentiles, a high, low and/or averages of performance indicators of other clinicians collectively, such as of a particular coaching type, in an HME or across the industry (e.g., all HMEs). Such comparison may help managers or decisionmakers within the HME provider understand a clinician' performance such as of particular coaching type in relation to the industry.

By way of further, the system 100 may process a filter criterion that identifies performance, such as an industry performance benchmark, based on device. For example, the system 100 may output a performance indicator (such as for comparison with other similar performance indicators associated with different devices) for particular patients (e.g., all patients, or all patients of a location, or of a particular HME, and/or with a particular clinician, and/or with a particular physician etc.) who use a particular device or device type. Such comparison may help managers or decisionmakers understand how and/or whether a device affects compliance such as across the industry, so they might consider buying certain devices that work well or well for them.

### 5.1.1.2.1 HME Provider Performance

In one example, the system 100 may receive a filter criterion 134 that identifies two or more HME providers. The system 100 may output a plurality of performance indicators such as to provide a performance comparison between the identified HME providers. In one example, performance of each HME provider may depend on a number or a percentage of compliant patients coached by the HME provider relative to the total number of patients coached by the HME provider.

The HME providers may have various sizes or have different patient volumes, ranging from small mom and pop shops to large organizations.

In one example, the system 100 may process a filter criterion 134 that identifies a patient volume. The system 100 may output an average compliance rate of HME providers of the identified patient volume. For example, the system 100 may generate an average compliance rate, e.g., 70%, of HME providers having 5000 patients. Moreover, the filter criterion for generating the HME provider performance indicator(s) may be further categorized by other details such as by device type and/or patient demographic information (e.g., age, sex etc.), to provide further characterization of the performance indicator for the HME such as for comparison with other HMEs.

In another example, the system 100 may process a filter criterion 134 that identifies a particular HME provider. In one instance, the performance indicator generated by the system 100 may indicate the number of compliant patients associated with the particular HME provider. In another instance, the system 100 may show a comparison of compliance rates between the identified HME provider and HME providers of a similar patient volume. For instance, the system 100 may generate a compliance rate of the identified HME provider in comparison to an average compliance rate of HME providers of a similar patient volume. The performance indicator generated by the system 100 may indicate a comparison between (1) a ratio between the number of compliant patients and a total number of patients associated with the identified HME provider and (2) an average ratio between a number of compliant patients and a total number of patients per HME provider of a patient volume similar to the identified HME provider identified. In one example, the processor 102 may generate a message when the ratio associated with the identified HME provider falls below the average ratio. The processor 102 may send the message to the identified HME provider.

In another example, the system 100 may show that the identified HME provider of 5000 patients has a compliance rate of 56%, whereas the benchmark for an average HME provider of a similar patient volume is 65%. As such, the identified HME provider performs below the industry average, suggesting a need for improvement within the identified HME provider.

In another example, the system 100 may process a filter criterion 134 that identifies two or more HME providers of a similar patient volume. The system 100 may output a performance comparison of the identified HME providers. For instance, when comparing two HME providers identified by two HME provider identifiers, the performance indicator generated by the system 100 may include: a ratio between a number of compliant patients and a total number of patients associated with a first HME provider identifier; and a ratio between a number of compliant patients and a total number of patients associated with a second HME provider identifier. An HME performance indicator, such when compared to an industry benchmark including other HMEs, such as an average of other HME indicators, can provide valuable information for the success of a particular HME for determining whether to establish relations with or even acquiring an HME.

In another example, the system 100 may process a filter criterion 134 searching for the top HME provider with the highest performance. The system 100 may output which HME provider has the highest compliance rate.

Performance or compliance results of HME providers may be accessible by physicians and/or insurers. Based on such information, a physician may choose which HME provider to partner with. Two different patients 114c and 114e may work with the same physician 120b, but with two different HME providers 110a and 110b. The system 100 may process a filter criterion 134 submitted by the physician 120b which identifies these HME providers 110a and 110b. The system 100 may output a performance comparison of the two HME providers 110a and 110b to the physician 120b. The HME provider with a higher performance may have a greater level of patient compliance success. Based on the performance of the HME providers 110a and 110b, the physician 120b may refer future patients to the HME provider with a higher performance.

Similarly, based on performance or compliance results of different HME providers, an insurer may choose which HME provider to establish a relationship. Two HME providers each may accept the same insurer, e.g., Blue Cross Blue Shield. In one example, the system 100 may process a filter criterion 134 submitted by the insurer which identifies these two HME providers. The system 100 may provide a performance comparison of the two HME providers to the insurer. The insurer may choose to partner with the HME having a higher performance.

### 5.1.1.2.2 Location Performance

The system 110 may generate performance benchmarks for different geographic locations. For example, the system 100 may indicate that virtual coaching setup works better in one location, e.g., Arizona, while in-person coaching setup works better in other locations, e.g., Florida.

In one example, the system 100 may process a filter criterion 134 that identifies a particular location. The system 100 may generate an average compliance rate of HME providers in the particular location. A physician may use such information to understand which HME provider at the particular location works best with the physician's patient.

In another example, the system 100 may process a filter criterion 134 that identifies two or more HMD providers at a particular location. The system 100 may generate a performance comparison of these identified HME providers at the particular location.

In one example, the system 100 may process a filter criterion 134 that identifies an HMD provider at a particular location. In one instance, the processor may determine a number of compliant patients associated with the identified HME provider at the particular location. In another instance, the system 100 may determine whether the identified HME provider of a particular location performs above or below an industry benchmark. The performance indicator generated by the system 100 may indicate a comparison between (1) a ratio between the number of compliant patients and a total number of patients associated with the identified HME provider at the particular location and (2) an average ratio between a number of compliant patients and a total number of patients coached at the particular location per HME provider. For example, if the identified HME provider has three offices in the particular location, e.g., three offices in the state of Florida, the system 100 may determine that the identified HME provider may have a cumulative compliance rate of 68% in that location. The system 100 may also determine that an average HME provider in the same location has a compliance rate of 65%. As a result, the system 100 may indicate that the identified HME provider has a better performance than the industry benchmark.

In another instance, the performance indicator generated by the system 100 may indicate a comparison between (1) a ratio between the number of compliant patients and a total number of patients associated with the identified HME provider at the particular location and (2) an average ratio between a number of compliant patients and a total number of patients coached at the particular location per HME provider of a patient volume similar to the identified HME provider.

### 5.1.1.2.3 Physician Performance

Physicians who work with the same HME provider may perform differently such that their patients have different compliance successes which may be detected by performance indicators of the present technology. Some physicians may have timely follow-up protocols with patients, while other physicians may not. Physicians of a higher performance may check with patients every few days to make sure that the patients are comfortable with the HME device, whereas physicians of a lower performance may contact patients less frequently. For example, after accepting new patients, some physicians may follow up with the new patients within a few days to make sure that the new patients are all set to use an HME device, whereas other physicians may not follow up with the new patients until a month later. As a result, performance of those other physicians is relatively low, if the new patients are expected to comply with the device usage requirement starting from the first two days.

In one example, the system 100 may process a filter criterion 134 that identifies an HME provider. The system 100 may generate a performance comparison of different physicians working with the identified HME provider. Such comparison may indicate which physicians work best with the given HME provider, and may also show indicate which physicians may benefit from improvement.

In another example, the system 100 may process a filter criterion 134 that identifies a particular physician. The system 100 may generate a performance indicator indicating a number of compliant patients, coached by the identified physician. In another instance, the system 100 may generate a performance comparison between the identified physician and the industry benchmark (e.g., an average physician performance). Such comparison may be accessed by a physician, so that the physician may obtain insights as to peer performance.

In one example, the system 100 may process a filter criterion 134 that requests performance of an identified physician across different HME providers. The system 100 may generate a performance comparison of the identified physician working with different HME providers. Using such information, the HME provider may improve its relationship with physicians having a lower performance. Further, using such information, the HME provider may also determine which physician it wants to partner with in the future.

In one example, the system 100 may process a filter criterion 134 that requests performance of an identified HME providers compared to other HME providers. The system 100 may generate a performance comparison of the identified HME relative to the performance of other HME providers. Using such information, a physician may determine which HME provider may be the best for establishing a relationship.

In one example, the system may process a filter criterion 134 that identifies a physician and an HME provider. The system 100 may generate a performance indicator that includes a ratio between a number of compliant patients and a total number of patients associated with the identified HME provider and coached by the identified physician. In another example, the filter criterion 134 may request a comparison between the physician's performance with the HME provider and an industry average. The performance indicator generated by the system 100 may indicate a comparison between (1) the ratio between a number of compliant patients and a total number of patients associated with the identified HME provider and coached by the identified physician and (2) an average ratio between a number of compliant patients and a total number of patients coached by the identified physician across the plurality of HME providers. The system 100 may output that the identified physician partnering with the identified HME provider has a compliance rate of 56%, whereas the same physician has an average compliance rate of 65% across different HME providers. Such comparison may encourage the identified HME provider to improve its relationship with the identified physician.

### 5.1.1.2.4 Insurer Performance

Different insurers may have different workflows, some of which may be efficient, while others may be inefficient, which may influence patient health as indicated by patient compliance. Thus, the system may generate the performance of a given insurer, and may compare performance across different insurers. Each insurer's performance may depend on a number or a percentage of compliant patients relative to a total number of patients associated with the insurer.

In one example, the system 100 may process a filter criterion 134 that identifies an insurer. The system 100 may output a performance indicator indicating a ratio between a number of compliant patients and a total number of patients associated with the identified insurer. In another instance, the performance indicator may indicate a comparison between (1) the ratio between a number of compliant patients and a total number of patients associated with the identified insurer and (2) an average ratio between a number of compliant patients and a total number of patients per insurer.

An insurer with a higher compliance rate may suggest that the insurer is more responsive to patients and/or generous, and may thus incentivize an HME provider to partner with the high-performance insurer and/or deepen its relationship with the high-performance insurer, so as to be able to help more patients such as by providing patient access to greater incentives to compliance such a with more generous reimbursements and/or discount benefits from the high-performance insurer.

### 5.1.2 Example Flow Charts

### 5.1.2.1 Flow Chart Related to Intra-Organizational Performance Benchmarks

FIG. 3 illustrates an example flow chart for a method for monitoring performance associated with HME. At 302, the memory 104 may store information for each of a plurality of patients. The stored information for each patient may include at least one of: (1) at least one coach identifier identifying at least one coach who coaches the patient, and (2) a geographic location where the patient receives coaching from the at least one coach. At 304, at least one processor(s) 102 may receive, from a plurality of HME devices, device usage of the plurality of patients. At 306, the at least one processor(s) 102 may determine whether at least one of the plurality of patients is a compliant patient by comparing the device usage of the at least one patient to a device usage requirement. At 308, the at least one processor(s) 102 may receive a filter criterion from a user. The filter criterion may include at least one selected coach identifier and/or at least one selected geographic location. At 310, the at least one processor(s) 102 may generate a performance indicator, for output to the user, based on a number of compliant patients whose information meets the filter criterion.

In one example, the device usage requirement may indicate a predetermined number of device operation hours.

In one example, the at least one coach of each patient may include a clinician and/or a physician.

In one example, the performance indicator may include a ratio between the number of compliant patients and a total number of the plurality of patients.

In one example, the number of compliant patients may be a number of compliant patients coached by at least one coach identified by the at least one selected coach identifier.

In one example, the performance indicator may indicate a progression of the number of compliant patients, coached by the at least one coach identified by the at least one selected coach identifier, over time.

In one example, the performance indicator may indicate a comparison between the number of compliant patients, coached by the at least one coach identified by the at least one selected coach identifier and an acceptable threshold.

In one example, the acceptable threshold may be an average number of compliant patients per coach.

In one example, the at least one processor(s) 102 may be configured to generate one or more messages or other electronic communication depending on different selectable rule(s). For example, performance messages, such as to report previously selected type(s) of performance indicator(s), may be generated periodically, such as monthly, weekly, etc. and/or may be generated based on detected condition(s) or change in condition(s) such as based on one or more comparisons between one or more performance indicators and one or more thresholds. Such messages may include the one or more selected performance indicator(s). For example, the system may be configured to generate a message when the number of compliant patients, coached by the at least one coach identified by the at least one selected coach identifier, based on a predetermined or user selectable threshold, such as when a related performance indicator falls below the acceptable threshold. Such messages may be configured to help clinicians, physicians, and/or HME for achieving their goals in performance and monitoring that achievement by triggering messages in relation to such a goal(s) that may be indicated by a predetermined goal threshold(s) or deviations from such threshold(s).

In one example, the at least one processor(s) 102 may send the generated message to the at least one coach identified by the at least one selected coach identifier.

In one example, the at least one coach identified by the at least one selected coach identifier may include at least one clinician and/or at least one physician.

In one example, the performance indicator may include an average number of patients per coach.

In one example, the number of compliant patients may be a number of compliant patients, coached at the at least one selected geographic location.

In one example, the number of compliant patients may be a number of compliant patients coached by at least one coach identified by the at least one selected coach identifier at the at least one selected geographic location.

In one example, the at least one selected geographic location may include at least two selected geographic locations. The performance indicator may include a ratio between a number of compliant patients and a total number of patients coached at one of the at least two selected geographic locations, and a ratio between a number of compliant patients and a total number of patients coached at another one of the at least two selected geographic locations.

In one example, the plurality of HME devices may be provided to the plurality of patients by an HME provider.

### 5.1.2.2 Flow Chart Related to Industry Performance Benchmarks

FIG. 4 illustrates an example flow chart of another method for monitoring performance associated with HME. At 402, the memory 104 may store information for each of a plurality of patients. The information for each patient may include at least one of: (1) an HME provider identifier identifying one of the plurality of HME providers associated with the patient, (2) a physician identifier identifying a physician who coaches the patient on using the HME device; (3) a geographic location where the patient receives coaching for using the HME device; and (4) an insurer identifier identifying an insurer associated with the patient.

At 404, at least one processor(s) 102 may receive, from a plurality of HME devices, device usage of the plurality of patients. The HME devices may be provided to the patients by a plurality of HME providers. At 406, the at least one processor(s) 102 may determine whether at least one of the plurality of patients is a compliant patient by comparing the device usage of the at least one patient to a device usage requirement. At 408, the at least one processor(s) 102 may receive a filter criterion, from a user. The filter criterion may include one or more of the following: at least one selected HME provider identifier, at least one selected physician identifier, at least one selected geographic location identifier, and at least one selected insurer identifier. At 410, the at least one processor(s) 102 may generate a performance indicator, for output to the user, based on a number of compliant patients whose information meets the filter criterion.

In one example, the plurality of HME providers may be of a plurality of patient volumes.

In one example, the at least one selected HME provider identifier may include at least two selected HME provider identifiers. The performance indicator may include a ratio between a number of compliant patients and a total number of patients associated with one of the at least two selected HME provider identifiers; and a ratio between a number of compliant patients and a total number of patients associated with another one of the at least two selected HME provider identifiers.

In one example, the number of compliant patients may be a number of compliant patients associated with at least one HME provider identified by the at least one selected HME provider identifier.

In one example, the performance indicator may indicate a comparison between (1) a ratio between the number of compliant patients and a total number of patients associated with the at least one HME provider identified by the at least one selected HME provider identifier and (2) an average ratio between a number of compliant patients and a total number of patients per HME provider of a patient volume similar to the at least one HME provider identified by the at least one selected HME provider identifier.

In one example, the at least one processor(s) 102 may be configured to generate a message when the ratio associated with the at least one HME provider identified by the at least one selected HME provider identifier falls below the average ratio.

In one example, the at least one processor(s) 102 may send the message to the at least one HME provider identified by the at least one selected HME provider identifier.

In one example, the number of compliant patients may be a number of compliant patients associated with at least one HME provider identified by the at least one selected HME provider identifier, and coached at the at least one selected geographic location.

In one example, the performance indicator may indicate a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider.

In one example, the performance indicator may indicate a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider of an patient volume similar to the at least one HME provider identified by the at least one selected HME provider identifier.

In one example, the number of compliant patients may be a number of compliant patients, coached by at least one physician identified by the at least one selected physician identifier

In one example, the performance indicator may include a ratio between a number of compliant patients and a total number of patients associated with at least one HME provider identified by the at least one HME provider identifier and coached by at least one physician identified by the at least one selected physician identifier. In one example, the performance indicator may indicate a comparison between (1) the ratio calculated above and (2) an average ratio between a number of compliant patients and a total number of patients coached by at least one physician identified by the at least one selected physician identifier across the plurality of HME providers.

In one example, the performance indicator may include a ratio between a number of compliant patients and a total number of patients associated with at least one insurer identified by the at least one insurer identifier.

In one example, the performance indicator may indicate a comparison between (1) the ratio and (2) an average ratio between a number of compliant patients and a total number of patients per insurer.

### 5.1.2.3 Example Processing Flow Chart

FIG. 5 illustrates yet another method for monitoring performance associated with HME. At 502, the memory 104 may store information for each of a plurality of patients. At 504, at least one processor(s) 102 may receive, from the plurality of HME devices, device usage of a plurality of patients. At 506, the at least one processor(s) 102 may determine whether at least one of the plurality of patients is a compliant patient by comparing the device usage of the at least one patient to a device usage requirement. At 508, the at least one processor(s) 102 may receive a filter criterion from a user. The filter criterion may concern the information. At 510, the at least one processor(s) 102 may generate a performance indicator, for output to the user, based on a number of compliant patients whose information meets the filter criterion.

In one example, the information for each patient may include at least one of: (1) at least one coach identifier identifying at least one coach who coaches the patient, and (2) a geographic location where the patient receives coaching from the at least one coach.

In one example, the filter criterion may include at least one selected coach identifier and/or at least one selected geographic location.

In one example, the HME devices may be provided to the patients by a plurality of HME providers.

In one example, the information for each patient may include at least one of: (1) an HME provider identifier identifying one of the plurality of HME providers associated with the patient, (2) a physician identifier identifying a physician who coaches the patient on using the HME device; (3) a geographic location where the patient receives coaching for using the HME device; and (4) an insurer identifier identifying an insurer associated with the patient.

In one example, the filter criterion may include one or more of the following: at least one selected HME provider identifier, at least one selected physician identifier, at least one selected geographic location identifier, and at least one selected insurer identifier.

In one example, the performance indicator may indicate an average number of compliant patients coached by an HME provider on a monthly basis.

### 5.1.3 Confidential Information

The system 100 may be configured to protect individual patient's personal information confidential, including, but not limited to, name, location and device usage information of the individual patient. Thus, when generating report or providing access to data of the system, the system may prevent users from accessing confidential patient information.

Similarly, the system may be configured to prevent data access to users with respect to information of competing entities, e.g., names of other HME providers or other physicians who are unrelated to a particular HME provider of a particular user of the system. Thus, when generating report or providing access to data of the system, the system may prevent users from accessing identifying information of other entities while still providing generalized cross industry information regarding the performance indicator(s) described herein.

### 5.1.4 Technical Advantages

The present technology may reduce burdens on healthcare systems with by providing improvements in data systems and data analytics that may lead to better therapy devices, healthier patients and/or better healthcare. The present technology, such as in relation to therapy device communications and data compilation systems with electronic communication systems (e.g., networked HME devices and server-based database(s)), improves data analytics processing and/or systematic computation analysis with computational machines (i.e., computers) so as to provide technological improvement that overcome the shortfalls of existing systems.

The present technology may provide insights as to patients' compliance regarding using HME devices, and identify problems that may affect patients' compliance, such as to increase overall compliance rates. Such problem identification, such as based on benchmarking showing proper instruction and coaching standards are being met so as to eliminate a potential human cause for low compliance, can help lead to identification of technical causes with particular designs of therapy devices that need to be improved for greater compliance and better therapy. Moreover, given such vast data as therapy usage by vast fleets of devices, the processes herein can derive and generate new indicators in rapid and readily understandable presentation to permit evaluation of otherwise complex and indecipherable information.

In one aspect, by performance benchmarks, such an intra-organizational performance benchmarks, the present technology may also suggest areas of improvements to an HME provider or that no improvements are necessary. Furthermore, by knowing its current compliance benchmarks, the HME provider may strive to improve areas of weakness, which increases patient treatment (i.e., operational use of therapy devices). For example, the HME provider may arrange a clinician or a location of a higher performance to share expertise with that of a lower performance.

In another aspect, by providing industry performance benchmarks, the present technology may provide insights to different users within the patient compliance cycle as to which parties to work with and how to work with them. For example, the present disclosure may reveal to an HME provider its current performance compared to the industry standard. In turn, the HME provider may take suitable actions to improve its performance so as to stay competitive in the industry, which again increases patient treatment (i.e., operational use of therapy devices).

### 5.2 RPT Device

In one form, each HME device may be an RPT device for treating a respiratory disorder, such as a positive airway pressure therapy device or a high flow therapy device. Referring to FIGS. 6A-6C, the RPT device 4000 may supply a flow of air to the patient 1000 via an air circuit 4170 and a patient interface 3000 or 3800.

The RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document. Such a device may provide a respiratory therapy, such a positive airway pressure therapy (e.g., CPAP, or bi-level CPAP, ventilator etc.) or high flow therapy, according to a flow control loop or pressure control loop.

In one form, the RPT device 4000 may be constructed and arranged to be capable of delivering a flow of air in a range of −20 L/min to +150 L/min while maintaining a positive pressure of at least 4 cmH2O, or at least 10 cmH2O, or at least 20 cmH2O.

With respect to FIG. 7A, the RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

As shown in FIG. 7C, the RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 5.2.1 RPT Device Mechanical & Pneumatic Components

An RPT device may comprise one or more of the following components in an integral unit. In an alternative form, one or more of the following components may be located as respective separate units.

### 5.2.1.1 Air Filter(s)

An RPT device in accordance with one form of the present technology may include an air filter 4110, or a plurality of air filters 4110.

In one form illustrated in FIG. 7B, an inlet air filter 4112 is located at the beginning of the pneumatic path upstream of a pressure generator 4140.

In one form illustrated in FIG. 7B, an outlet air filter 4114, for example an antibacterial filter, is located between an outlet of the pneumatic block 4020 and a patient interface 3000 or 3800.

### 5.2.1.2 Muffler(s)

An RPT device in accordance with one form of the present technology may include a muffler 4120, or a plurality of mufflers 4120.

In one form of the present technology (see e.g., FIG. 7B), an inlet muffler 4122 is located in the pneumatic path upstream of a pressure generator 4140.

In one form of the present technology, an outlet muffler 4124 is located in the pneumatic path between the pressure generator 4140 and a patient interface 3000 or 3800.

### 5.2.1.3 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 may be under the control of the therapy device controller 4240.

In other forms, a pressure generator 4140 may be a piston-driven pump, a pressure regulator connected to a high pressure source (e.g. compressed air reservoir), or a bellows.

### 5.2.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology (see e.g., FIG. 7B), one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000 or 3800.

In one form, a signal from a transducer 4270 may be filtered, such as by low-pass, high-pass or band-pass filtering.

### 5.2.1.4.1 Flow Rate Sensor

A flow rate sensor 4274 in accordance with the present technology may be based on a differential pressure transducer, for example, an SDP600 Series differential pressure transducer from SENSIRION.

In one form, a signal generated by the flow rate sensor 4274 and representing a flow rate is received by the central controller 4230.

### 5.2.1.4.2 Pressure Sensor

A pressure sensor 4272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal generated by the pressure sensor 4272 and representing a pressure is received by the central controller 4230.

### 5.2.1.4.3 Motor Speed Transducer

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor

### 5.2.1.5 Anti-Spill Back Valve

As shown in FIG. 7B, one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 5.2.2 RPT Device Electrical Components

### 5.2.2.1 Power Supply

A power supply 4210 may be located internal or external of the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

### 5.2.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form, the input device 4220 may be constructed and arranged to allow a person to select a value and/or a menu option.

### 5.2.2.3 Central Controller

In one form of the present technology, the central controller 4230 is one or a plurality of processors suitable to control an RPT device 4000. The central controller 4230 is show in FIG. 7C.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and/or the humidifier 5000.

The central controller 4230 may be configured to provide output signal(s) to one or more of an output device 4290, a pressure generator 4140, a therapy device controller 4240, a data communication interface 4280, and/or the humidifier 5000.

In some forms of the present technology, the central controller 4230 is configured to implement the one or more methodologies described herein, such as the one or more algorithms 4300 which may be implemented with processor-control instructions, expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.2.2.4 Clock

The RPT device 4000 may include a clock 4232 that is connected to the central controller 4230.

### 5.2.2.5 Therapy Device Controller

Referring to FIG. 7D, in one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 5.2.2.6 Protection Circuits

The one or more protection circuits 4250 in accordance with the present technology may comprise an electrical protection circuit, a temperature and/or pressure safety circuit.

### 5.2.2.7 Memory

In accordance with one form of the present technology the RPT device 4000 includes memory 4260, e.g., non-volatile memory. In some forms, memory 4260 may include battery powered static RAM. In some forms, memory 4260 may include volatile RAM.

Memory 4260 may be located on the PCBA 4202. Memory 4260 may be in the form of EEPROM, or NAND flash.

Additionally, or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300.

### 5.2.2.8 Data Communication Systems

In one form of the present technology, a data communication interface 4280 is provided, and is connected to the central controller 4230 (see e.g., FIG. 7C). Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g., via Ethernet, or optical fibre) or a wireless protocol (e.g., CDMA, GSM, LTE) to connect to the Internet.

In one form, local external communication network 4284 utilises one or more communication standards, such as Bluetooth, or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 5.2.2.9 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 5.2.2.9.1 Display Driver

A display driver 4292 receives as an input the characters, symbols, or images intended for display on the display 4294, and converts them to commands that cause the display 4294 to display those characters, symbols, or images.

### 5.2.2.9.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 5.2.3 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 5.2.3.1 Pre-Processing Module

A pre-processing module 4310 in accordance with one form of the present technology receives as an input a signal from a transducer 4270, for example a flow rate sensor 4274 or pressure sensor 4272, and performs one or more process steps to calculate one or more output values that will be used as an input to another module, for example a therapy engine module 4320.

In one form of the present technology, the output values include the interface pressure Pm, the vent flow rate Qv, the respiratory flow rate Qr, and the leak flow rate Ql.

In various forms of the present technology, the pre-processing module 4310 comprises one or more of the following algorithms: interface pressure estimation 4312, vent flow rate estimation 4314, leak flow rate estimation 4316, and respiratory flow rate estimation 4318.

### 5.2.3.1.1 Interface Pressure Estimation

In one form of the present technology, an interface pressure estimation algorithm 4312 receives as inputs a signal from the pressure sensor 4272 indicative of the pressure in the pneumatic path proximal to an outlet of the pneumatic block (the device pressure Pd) and a signal from the flow rate sensor 4274 representative of the flow rate of the airflow leaving the RPT device 4000 (the device flow rate Qd). The device flow rate Qd, absent any supplementary gas 4180, may be used as the total flow rate Ot. The interface pressure algorithm 4312 estimates the pressure drop ΔP through the air circuit 4170. The dependence of the pressure drop ΔP on the total flow rate Qt may be modelled for the particular air circuit 4170 by a pressure drop characteristic ΔP(Q). The interface pressure estimation algorithm, 4312 then provides as an output an estimated pressure, Pm, in the patient interface 3000 or 3800. The pressure, Pm, in the patient interface 3000 or 3800 may be estimated as the device pressure Pd minus the air circuit pressure drop ΔP.

### 5.2.3.1.2 Vent Flow Rate Estimation

In one form of the present technology, a vent flow rate estimation algorithm 4314 receives as an input an estimated pressure, Pm, in the patient interface 3000 or 3800 from the interface pressure estimation algorithm 4312 and estimates a vent flow rate of air, Qv, from a vent 3400 in a patient interface 3000 or 3800. The dependence of the vent flow rate Qv on the interface pressure Pm for the particular vent 3400 in use may be modelled by a vent characteristic Qv(Pm).

### 5.2.3.1.3 Leak Flow Rate Estimation

In one form of the present technology, a leak flow rate estimation algorithm 4316 receives as an input a total flow rate, Qt, and a vent flow rate Ov, and provides as an output an estimate of the leak flow rate Ql. In one form, the leak flow rate estimation algorithm estimates the leak flow rate Ql by calculating an average of the difference between total flow rate Qt and vent flow rate Ov over a period sufficiently long to include several breathing cycles, e.g. about 10 seconds.

In one form, the leak flow rate estimation algorithm 4316 receives as an input a total flow rate Qt, a vent flow rate Qv, and an estimated pressure, Pm, in the patient interface 3000 or 3800, and provides as an output a leak flow rate Ql, by calculating a leak conductance, and determining a leak flow rate Ql to be a function of leak conductance and pressure, Pm. Leak conductance is calculated as the quotient of low pass filtered non-vent flow rate equal to the difference between total flow rate Qt and vent flow rate Qv, and low pass filtered square root of pressure Pm, where the low pass filter time constant has a value sufficiently long to include several breathing cycles, e.g. about 10 seconds. The leak flow rate Ql may be estimated as the product of leak conductance and a function of pressure, Pm.

### 5.2.3.1.4 Respiratory Flow Rate Estimation

In one form of the present technology, a respiratory flow rate estimation algorithm 4318 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

### 5.2.3.2 Therapy Engine Module

In one form of the present technology, a therapy engine module 4320 receives as inputs one or more of a pressure, Pm, in a patient interface 3000 or 3800, and a respiratory flow rate of air to a patient, Qr, and provides as an output one or more therapy parameters.

In one form of the present technology, a therapy parameter is a treatment pressure Pt.

In one form of the present technology, therapy parameters are one or more of an amplitude of a pressure variation, a base pressure, and a target ventilation.

In various forms, the therapy engine module 4320 comprises one or more of the following algorithms: phase determination 4321, waveform determination 4322, ventilation determination 4323, inspiratory flow limitation determination 4324, apnea/hypopnea determination 4325, snore determination 4326, airway patency determination 4327, target ventilation determination 4328, and therapy parameter determination 4329.

### 5.2.3.2.1 Phase Determination

In one form of the present technology, the RPT device 4000 does not determine phase.

In one form of the present technology, a phase determination algorithm 4321 receives as an input a signal indicative of respiratory flow rate, Qr, and provides as an output a phase Φ of a current breathing cycle of a patient 1000.

In some forms, known as discrete phase determination, the phase output Φ is a discrete variable. One implementation of discrete phase determination provides a bi-valued phase output Φ with values of either inhalation or exhalation, for example represented as values of 0 and 0.5 revolutions respectively, upon detecting the start of spontaneous inhalation and exhalation respectively. RPT devices 4000 that “trigger” and “cycle” effectively perform discrete phase determination, since the trigger and cycle points are the instants at which the phase changes from exhalation to inhalation and from inhalation to exhalation, respectively. In one implementation of bi-valued phase determination, the phase output Φ is determined to have a discrete value of 0 (thereby “triggering” the RPT device 4000) when the respiratory flow rate Qr has a value that exceeds a positive threshold, and a discrete value of 0.5 revolutions (thereby “cycling” the RPT device 4000) when a respiratory flow rate Qr has a value that is more negative than a negative threshold. The inhalation time Ti and the exhalation time Te may be estimated as typical values over many respiratory cycles of the time spent with phase Φ equal to 0 (indicating inspiration) and 0.5 (indicating expiration) respectively.

Another implementation of discrete phase determination provides a tri-valued phase output Φ with a value of one of inhalation, mid-inspiratory pause, and exhalation.

In other forms, known as continuous phase determination, the phase output Φ is a continuous variable, for example varying from 0 to 1 revolutions, or 0 to 2π radians. RPT devices 4000 that perform continuous phase determination may trigger and cycle when the continuous phase reaches 0 and 0.5 revolutions, respectively. In one implementation of continuous phase determination, a continuous value of phase Φ is determined using a fuzzy logic analysis of the respiratory flow rate Qr. A continuous value of phase determined in this implementation is often referred to as “fuzzy phase”. In one implementation of a fuzzy phase determination algorithm 4321, the following rules are applied to the respiratory flow rate Qr:
1. If Qr is zero and increasing fast then Φ is 0 revolutions.
2. If Qr is large positive and steady then Φ is 0.25 revolutions.
3. If Qr is zero and falling fast, then Φ is 0.5 revolutions.
4. If Qr is large negative and steady then Φ is 0.75 revolutions.
5. If Qr is zero and steady and the 5-second low-pass filtered absolute value of Qr is large then Φ is 0.9 revolutions.
6. If Qr is positive and the phase is expiratory, then Φ is 0 revolutions.
7. If Qr is negative and the phase is inspiratory, then Φ is 0.5 revolutions.
8. If the 5-second low-pass filtered absolute value of Qr is large, Φ is increasing at a steady rate equal to the patient's breathing rate, low-pass filtered with a time constant of 20 seconds.

The output of each rule may be represented as a vector whose phase is the result of the rule and whose magnitude is the fuzzy extent to which the rule is true. The fuzzy extent to which the respiratory flow rate is “large”, “steady”, etc. is determined with suitable membership functions. The results of the rules, represented as vectors, are then combined by some function such as taking the centroid. In such a combination, the rules may be equally weighted, or differently weighted.

In another implementation of continuous phase determination, the phase Φ is first discretely estimated from the respiratory flow rate Qr as described above, as are the inhalation time Ti and the exhalation time Te. The continuous phase Φ at any instant may be determined as the half the proportion of the inhalation time Ti that has elapsed since the previous trigger instant, or 0.5 revolutions plus half the proportion of the exhalation time Te that has elapsed since the previous cycle instant (whichever instant was more recent).

### 5.2.3.2.2 Waveform Determination

In one form of the present technology, the therapy parameter determination algorithm 4329 provides an approximately constant treatment pressure throughout a respiratory cycle of a patient.

In other forms of the present technology, the therapy control module 4330 controls the pressure generator 4140 to provide a treatment pressure Pt that varies as a function of phase Φ of a respiratory cycle of a patient according to a waveform template Π(Φ).

In one form of the present technology, a waveform determination algorithm 4322 provides a waveform template Π(Φ) with values in the range [0, 1] on the domain of phase values Φ provided by the phase determination algorithm 4321 to be used by the therapy parameter determination algorithm 4329.

In one form, suitable for either discrete or continuously-valued phase, the waveform template Π(Φ) is a square-wave template, having a value of 1 for values of phase up to and including 0.5 revolutions, and a value of 0 for values of phase above 0.5 revolutions.

In one form, suitable for continuously-valued phase, the waveform template Π(Φ) comprises two smoothly curved portions, namely a smoothly curved (e.g. raised cosine) rise from 0 to 1 for values of phase up to 0.5 revolutions, and a smoothly curved (e.g. exponential) decay from 1 to 0 for values of phase above 0.5 revolutions. In one form, suitable for continuously-valued phase, the waveform template Π(Φ) is based on a square wave, but with a smooth rise from 0 to 1 for values of phase up to a “rise time” that is less than 0.5 revolutions, and a smooth fall from 1 to 0 for values of phase within a “fall time” after 0.5 revolutions, with a “fall time” that is less than 0.5 revolutions.

In some forms of the present technology, the waveform determination algorithm 4322 selects a waveform template Π(Φ) from a library of waveform templates, dependent on a setting of the RPT device. Each waveform template Π(Φ) in the library may be provided as a lookup table of values Π against phase values Φ. In other forms, the waveform determination algorithm 4322 computes a waveform template Π(Φ) “on the fly” using a predetermined functional form, possibly parametrised by one or more parameters (e.g. time constant of an exponentially curved portion). The parameters of the functional form may be predetermined or dependent on a current state of the patient 1000.

In some forms of the present technology, suitable for discrete bi-valued phase of either inhalation (Φ=0 revolutions) or exhalation (Φ=0.5 revolutions), the waveform determination algorithm 4322 computes a waveform template Π “on the fly” as a function of both discrete phase Φ and time t measured since the most recent trigger instant. In one such form, the waveform determination algorithm 4322 computes the waveform template Π(Φ, t) in two portions (inspiratory and expiratory) as follows:

$$Π ( Φ , t ) = \{ \begin{matrix}{Π}_{i} ( t ) , & Φ = 0 \\ {Π}_{e} ( t - {T}_{i} ) , & Φ = 0.5\end{matrix}$$

where Πᵢ(t) and Πₑ(t) are inspiratory and expiratory portions of the waveform template Π(Φ, t). In one such form, the inspiratory portion Πᵢ(t) of the waveform template is a smooth rise from 0 to 1 parametrised by a rise time, and the expiratory portion Πₑ(t) of the waveform template is a smooth fall from 1 to 0 parametrised by a fall time.

### 5.2.3.2.3 Ventilation Determination

In one form of the present technology, a ventilation determination algorithm 4323 receives an input a respiratory flow rate Qr, and determines a measure indicative of current patient ventilation, Vent.

In some implementations, the ventilation determination algorithm 4323 determines a measure of ventilation Vent that is an estimate of actual patient ventilation. One such implementation is to take half the absolute value of respiratory flow rate, Qr, optionally filtered by low-pass filter such as a second order Bessel low-pass filter with a corner frequency of 0.11 Hz.

In other implementations, the ventilation determination algorithm 4323 determines a measure of ventilation Vent that is broadly proportional to actual patient ventilation. One such implementation estimates peak respiratory flow rate Qpeak over the inspiratory portion of the cycle. This and many other procedures involving sampling the respiratory flow rate Qr produce measures which are broadly proportional to ventilation, provided the flow rate waveform shape does not vary very much (here, the shape of two breaths is taken to be similar when the flow rate waveforms of the breaths normalised in time and amplitude are similar). Some simple examples include the median positive respiratory flow rate, the median of the absolute value of respiratory flow rate, and the standard deviation of flow rate. Arbitrary linear combinations of arbitrary order statistics of the absolute value of respiratory flow rate using positive coefficients, and even some using both positive and negative coefficients, are approximately proportional to ventilation. Another example is the mean of the respiratory flow rate in the middle K proportion (by time) of the inspiratory portion, where 0<K<1. There is an arbitrarily large number of measures that are exactly proportional to ventilation if the flow rate shape is constant.

### 5.2.3.2.4 Determination of Inspiratory Flow Limitation

In one form of the present technology, the central controller 4230 executes an inspiratory flow limitation determination algorithm 4324 for the determination of the extent of inspiratory flow limitation.

In one form, the inspiratory flow limitation determination algorithm 4324 receives as an input a respiratory flow rate signal Qr and provides as an output a metric of the extent to which the inspiratory portion of the breath exhibits inspiratory flow limitation.

In one form of the present technology, the inspiratory portion of each breath is identified by a zero-crossing detector. A number of evenly spaced points (for example, sixty-five), representing points in time, are interpolated by an interpolator along the inspiratory flow rate-time curve for each breath. The curve described by the points is then scaled by a scalar to have unity length (duration/period) and unity area to remove the effects of changing breathing rate and depth. The scaled breaths are then compared in a comparator with a pre-stored template representing a normal unobstructed breath. Breaths deviating by more than a specified threshold (typically 1 scaled unit) at any time during the inspiration from this template, such as those due to coughs, sighs, swallows and hiccups, as determined by a test element, are rejected. For non-rejected data, a moving average of the first such scaled point is calculated by the central controller 4230 for the preceding several inspiratory events. This is repeated over the same inspiratory events for the second such point, and so on. Thus, for example, sixty-five scaled data points are generated by the central controller 4230, and represent a moving average of the preceding several inspiratory events, e.g., three events. The moving average of continuously updated values of the (e.g., sixty-five) points are hereinafter called the “scaled flow rate”, designated as Qs(t). Alternatively, a single inspiratory event can be utilised rather than a moving average.

From the scaled flow rate, two shape factors relating to the determination of partial obstruction may be calculated.

Shape factor 1 is the ratio of the mean of the middle (e.g. thirty-two) scaled flow rate points to the mean overall (e.g. sixty-five) scaled flow rate points. Where this ratio is in excess of unity, the breath will be taken to be normal. Where the ratio is unity or less, the breath will be taken to be obstructed. A ratio of about 1.17 is taken as a threshold between partially obstructed and unobstructed breathing, and equates to a degree of obstruction that would permit maintenance of adequate oxygenation in a typical patient.

Shape factor 2 is calculated as the RMS deviation from unit scaled flow rate, taken over the middle (e.g. thirty-two) points. An RMS deviation of about 0.2 units is taken to be normal. An RMS deviation of zero is taken to be a totally flow-limited breath. The closer the RMS deviation to zero, the breath will be taken to be more flow limited.

Shape factors 1 and 2 may be used as alternatives, or in combination. In other forms of the present technology, the number of sampled points, breaths and middle points may differ from those described above. Furthermore, the threshold values can be other than those described.

### 5.2.3.2.5 Determination of Apneas and Hypopneas

In one form of the present technology, the central controller 4230 executes an apnea/hypopnea determination algorithm 4325 for the determination of the presence of apneas and/or hypopneas.

In one form, the apnea/hypopnea determination algorithm 4325 receives as an input a respiratory flow rate signal Qr and provides as an output a flag that indicates that an apnea or a hypopnea has been detected.

In one form, an apnea will be said to have been detected when a function of respiratory flow rate Qr falls below a flow rate threshold for a predetermined period of time. The function may determine a peak flow rate, a relatively short-term mean flow rate, or a flow rate intermediate of relatively short-term mean and peak flow rate, for example an RMS flow rate. The flow rate threshold may be a relatively long-term measure of flow rate.

In one form, a hypopnea will be said to have been detected when a function of respiratory flow rate Qr falls below a second flow rate threshold for a predetermined period of time. The function may determine a peak flow, a relatively short-term mean flow rate, or a flow rate intermediate of relatively short-term mean and peak flow rate, for example an RMS flow rate. The second flow rate threshold may be a relatively long-term measure of flow rate. The second flow rate threshold is greater than the flow rate threshold used to detect apneas.

### 5.2.3.2.6 Determination of Snore

In one form of the present technology, the central controller 4230 executes one or more snore determination algorithms 4326 for the determination of the extent of snore.

In one form, the snore determination algorithm 4326 receives as an input a respiratory flow rate signal Qr and provides as an output a metric of the extent to which snoring is present.

The snore determination algorithm 4326 may comprise the step of determining the intensity of the flow rate signal in the range of 30-300 Hz. Further, the snore determination algorithm 4326 may comprise a step of filtering the respiratory flow rate signal Qr to reduce background noise, e.g., the sound of airflow in the system from the blower.

### 5.2.3.2.7 Determination of Airway Patency

In one form of the present technology, the central controller 4230 executes one or more airway patency determination algorithms 4327 for the determination of the extent of airway patency.

In one form, the airway patency determination algorithm 4327 receives as an input a respiratory flow rate signal Qr, and determines the power of the signal in the frequency range of about 0.75 Hz and about 3 Hz. The presence of a peak in this frequency range is taken to indicate an open airway. The absence of a peak is taken to be an indication of a closed airway.

In one form, the frequency range within which the peak is sought is the frequency of a small forced oscillation in the treatment pressure Pt. In one implementation, the forced oscillation is of frequency 2 Hz with amplitude about 1 cmH₂O.

In one form, airway patency determination algorithm 4327 receives as an input a respiratory flow rate signal Or, and determines the presence or absence of a cardiogenic signal. The absence of a cardiogenic signal is taken to be an indication of a closed airway.

### 5.2.3.2.8 Determination of Target Ventilation

In one form of the present technology, the central controller 4230 takes as input the measure of current ventilation, Vent, and executes one or more target ventilation determination algorithms 4328 for the determination of a target value Vtgt for the measure of ventilation.

In some forms of the present technology, there is no target ventilation determination algorithm 4328, and the target value Vtgt is predetermined, for example by hard-coding during configuration of the RPT device 4000 or by manual entry through the input device 4220.

In other forms of the present technology, such as adaptive servo-ventilation (ASV), the target ventilation determination algorithm 4328 computes a target value Vtgt from a value Vtyp indicative of the typical recent ventilation of the patient.

In some forms of adaptive servo-ventilation, the target ventilation Vtgt is computed as a high proportion of, but less than, the typical recent ventilation Vtyp. The high proportion in such forms may be in the range (80%, 100%), or (85%, 95%), or (87%, 92%).

In other forms of adaptive servo-ventilation, the target ventilation Vtgt is computed as a slightly greater than unity multiple of the typical recent ventilation Vtyp.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the target ventilation determination algorithm 4328, the recent history is of the order of several minutes, but in any case should be longer than the timescale of Cheyne-Stokes waxing and waning cycles. The target ventilation determination algorithm 4328 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 5.2.3.2.9 Determination of Therapy Parameters

In some forms of the present technology, the central controller 4230 executes one or more therapy parameter determination algorithms 4329 for the determination of one or more therapy parameters using the values returned by one or more of the other algorithms in the therapy engine module 4320.

In one form of the present technology, the therapy parameter is an instantaneous treatment pressure Pt. In one implementation of this form, the therapy parameter determination algorithm 4329 determines the treatment pressure Pt using the equation

$$Pt = A Π ( Φ , t ) + {P}_{0} \qquad ( 1 )$$

where:
A is the amplitude,
Π(Φ, t) is the waveform template value (in the range 0 to 1) at the current value Φ of phase and t of time, and
P₀ is a base pressure.

If the waveform determination algorithm 4322 provides the waveform template Π(Φ, t) as a lookup table of values Π indexed by phase Φ, the therapy parameter determination algorithm 4329 applies equation (1) by locating the nearest lookup table entry to the current value Φ of phase returned by the phase determination algorithm 4321, or by interpolation between the two entries straddling the current value Φ of phase.

The values of the amplitude A and the base pressure P₀ may be set by the therapy parameter determination algorithm 4329 depending on the chosen respiratory pressure therapy mode in the manner described below.

### 5.2.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of air whose interface pressure Pm at the patient interface 3000 or 3800 is equal to the treatment pressure Pt.

### 5.2.3.4 Detection of Fault Conditions

In one form of the present technology, the central controller 4230 executes one or more methods 4340 for the detection of fault conditions. The fault conditions detected by the one or more methods 4340 may include at least one of the following:
Power failure (no power, or insufficient power)
Transducer fault detection
Failure to detect the presence of a component
Operating parameters outside recommended ranges (e.g., pressure, flow rate, temperature, PaO2)
Failure of a test alarm to generate a detectable alarm signal.

Upon detection of the fault condition, the corresponding algorithm 4340 signals the presence of the fault by one or more of the following:
Initiation of an audible, visual &/or kinetic (e.g., vibrating) alarm
Sending a message to an external device
Logging of the incident

### 5.3 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases, there may be separate limbs of the circuit for inhalation and exhalation. In other cases, a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 5.4 Respiratory Therapy Modes

Various respiratory therapy modes may be implemented by the disclosed respiratory therapy system, including but not limited to, CPAP therapy, bi-level therapy and high flow therapy, among other possibilities.

### 5.5 Glossary

For the purposes of the present technology disclosure, in certain forms of the present technology, one or more of the following definitions may apply. In other forms of the present technology, alternative definitions may apply.

### 5.5.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Od, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar~0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 5.5.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Or): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

Inhalation Time (Ti): The duration of the inspiratory portion of the respiratory flow rate waveform.

Exhalation Time (Te): The duration of the expiratory portion of the respiratory flow rate waveform.

Total Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.6 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means +/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, “criterion” and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior disclosure. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples or implementations, it is to be understood that these examples or implementations are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and implementations and that other arrangements may be devised without departing from the spirit and scope of the technology such as by combining any of the aspects of any of the examples or implementations with any of the aspects of any of the other examples or implementations. Examples or implementations and the aspects thereof may be considered in relation to the following.

Example 1. A system for monitoring performance associated with home medical equipment (HME), comprising:
a non-transitory computer-readable medium that stores information for each of a plurality of patients, including, for each patient, one or more of: (a) at least one coach identifier identifying at least one coach who coaches the patient, and (b) a geographic location where the patient receives coaching from the at least one coach;
one or more processors configured to:
access device usage data of the plurality of patients that is repeatedly received
from a plurality of HME devices;
determine whether each of the plurality of patients is a compliant patient by comparing the device usage data to one or more device usage requirements;
receive a filter criterion, from a user, the filter criterion including at least one selected coach identifier and/or at least one selected geographic location; and
generate a performance indicator, for output to the user, based on a number of compliant patients whose information meets the filter criterion.

Example 2. The system of example 1, wherein a device usage data requirement of the one or more device usage requirements indicates a predetermined number of device operation hours.

Example 3. The system of any one of examples 1 to 2, wherein the at least one coach of each patient includes a clinician and/or a physician.

Example 4. The system of any one of examples 1 to 3, wherein the performance indicator includes a ratio between the number of compliant patients and a total number of the plurality of patients.

Example 5. The system of any one of examples 1 to 4, wherein the number of compliant patients is a number of compliant patients, coached by at least one coach identified by the at least one selected coach identifier.

Example 6. The system of example 5, wherein the performance indicator indicates a progression of the number of compliant patients, coached by the at least one coach identified by the at least one selected coach identifier, over time.

Example 7. The system of example 5, wherein the performance indicator indicates a comparison between the number of compliant patients, coached by the at least one coach identified by the at least one selected coach identifier and an acceptable threshold.

Example 8. The system of example 7, wherein the acceptable threshold is an average number of compliant patients per coach.

Example 9. The system of example 7, wherein the one or more processors is configured to generate a message when the number of compliant patients, coached by the at least one coach identified by the at least one selected coach identifier, falls below the acceptable threshold.

Example 10. The system of example 9, wherein the one or more processors is configured to send the generated message to the at least one coach identified by the at least one selected coach identifier.

Example 11. The system of example 5, wherein the at least one coach identified by the at least one selected coach identifier includes at least one clinician and/or at least one physician.

Example 12. The system of any one of examples 1 to 11, wherein the performance indicator includes an average number of patients per coach.

Example 13. The system of any one of examples 1 to 12, wherein the number of compliant patients is a number of compliant patients, coached at the at least one selected geographic location.

Example 14. The system of any one of examples 1 to 12, wherein the number of compliant patients is a number of compliant patients, coached by at least one coach identified by the at least one selected coach identifier at the at least one selected geographic location.

Example 15. The system of any one of examples 1 to 14, wherein the at least one selected geographic location includes at least two selected geographic locations, and wherein the performance indicator includes:
a ratio between a number of compliant patients and a total number of patients coached at one of the at least two selected geographic locations; and
a ratio between a number of compliant patients and a total number of patients coached at another one of the at least two selected geographic locations.

Example 16. The system of any one of examples 1 to 15, wherein the plurality of HME devices are provided to the plurality of patients by an HME provider.

Example 17. A system for monitoring performance associated with home medical equipment (HME), comprising:
a non-transitory computer-readable medium that stores information for each of a plurality of patients, including, for each patient, one or more of: (a) an HME provider identifier identifying one of a plurality of HME providers, wherein the one is associated with the patient, (b) a physician identifier identifying a physician who coaches the patient on using a HME device; (c) a geographic location where the patient receives coaching for using the HME device; and (d) an insurer identifier identifying an insurer associated with the patient; and
one or more processors configured to:
access device usage data of the plurality of patients that is repeatedly received from a plurality of HME devices, the HME devices being provided to the patients by a plurality of HME providers;
determine whether each of the plurality of patients is a compliant patient by comparing the device usage data to one or more device usage requirements;
receive a filter criterion, from a user, the filter criterion including one or more of the following: at least one selected HME provider identifier, at least one selected physician identifier, at least one selected geographic location, and at least one selected insurer identifier; and
generate a performance indicator, for output to the user, based on a number of compliant patients whose information meets the filter criterion.

Example 18. The system of example 17, wherein each of the plurality of HME providers have a plurality of patients.

Example 19. The system of any one of examples 17 to 18, wherein the at least one selected HME provider identifier includes at least two selected HME provider identifiers, and wherein the performance indicator includes:
a ratio between a number of compliant patients and a total number of patients
associated with one of the at least two selected HME provider identifiers; and
a ratio between a number of compliant patients and a total number of patients associated with another one of the at least two selected HME provider identifiers.

Example 20. The system of any one of examples 17 to 19, wherein the number of compliant patients is a number of compliant patients associated with at least one HME provider identified by the at least one selected HME provider identifier.

Example 21. The system of example 20, wherein the performance indicator indicates a comparison between (1) a ratio between the number of compliant patients and a total number of patients associated with the at least one HME provider identified by the at least one selected HME provider identifier and (2) an average ratio between a number of compliant patients and a total number of patients per HME provider of a patient volume similar to the at least one HME provider identified by the at least one selected HME provider identifier.

Example 22. The system of example 21, wherein the one or more processors is configured to generate a message when the ratio associated with the at least one HME provider identified by the at least one selected HME provider identifier falls below the average ratio.

Example 23. The system of example 22, wherein the one or more processors is configured to send the message to the at least one HME provider identified by the at least one selected HME provider identifier.

Example 24. The system of any one of examples 17 to 23, wherein the number of compliant patients is a number of compliant patients associated with at least one HME provider identified by the at least one selected HME provider identifier, and coached at the at least one selected geographic location.

Example 25. The system of example 24, wherein the performance indicator indicates a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider.

Example 26. The system of example 24, wherein the performance indicator indicates a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider of a patient volume similar to the at least one HME provider identified by the at least one selected HME provider identifier.

Example 27. The system of any one of examples 17 to 26, wherein the number of compliant patients is a number of compliant patients, coached by at least one physician identified by the at least one selected physician identifier.

Example 28. The system of any one of examples 17 to 27, wherein the performance indicator includes a ratio between a number of compliant patients and a total number of patients associated with at least one HME provider identified by the at least one HME provider identifier and coached by at least one physician identified by the at least one selected physician identifier.

Example 29. The system of example 28, wherein the performance indicator indicates a comparison between (1) the ratio and (2) an average ratio between a number of compliant patients and a total number of patients coached by at least one physician identified by the at least one selected physician identifier across the plurality of HME providers.

Example 30. The system of any one of examples 17 to 27, wherein the performance indicator includes a ratio between a number of compliant patients and a total number of patients associated with at least one insurer identified by the at least one insurer identifier.

Example 31. The system of example 30, wherein the performance indicator indicates a comparison between (1) the ratio and (2) an average ratio between a number of compliant patients and a total number of patients per insurer.

Example 32. A method for monitoring performance associated with home medical equipment (HME), comprising:
storing, by a non-transitory computer-readable medium, information for each of a plurality of patients, wherein the information for each patient includes at least one of: (a) at least one coach identifier identifying at least one coach who coaches the patient, and (b) a geographic location where the patient receives coaching from the at least one coach;
accessing, by one or more processors, device usage data of the plurality of patients that is received from a plurality of HME devices;
determining, by the one or more processors, whether each of the plurality of patients is a compliant patient by comparing the device usage data to one or more device usage requirements;
receiving, by the one or more processors, a filter criterion, from a user, the filter criterion including at least one selected coach identifier and/or at least one selected geographic location; and
generating, by the one or more processors, a performance indicator, for output to the user, based on a number of compliant patients whose information meets the filter criterion.

Example 33. The method of example 32, wherein the device usage requirement indicates a predetermined number of device operation hours.

Example 34. The method of any one of examples 32 to 33, wherein the at least one coach of each patient includes a clinician and/or a physician.

Example 35. The method of any one of examples 32 to 34, wherein the performance indicator includes a ratio between the number of compliant patients and a total number of the plurality of patients.

Example 36. The method of any one of examples 32 to 35, wherein the number of compliant patients is a number of compliant patients, coached by at least one coach identified by the at least one selected coach identifier.

Example 37. The method of example 36, wherein the performance indicator indicates a progression of the number of compliant patients, coached by the at least one coach identified by the at least one selected coach identifier, over time.

Example 38. The method of example 36, wherein the performance indicator indicates a comparison between the number of compliant patients, coached by the at least one coach identified by the at least one selected coach identifier and an acceptable threshold.

Example 39. The method of example 38, wherein the acceptable threshold is an average number of compliant patients per coach.

Example 40. The method of example 38, further comprising:
generating, by the one or more processors, a message when the number of compliant patients, coached by the at least one coach identified by the at least one selected coach identifier, falls below the acceptable threshold.

Example 41. The method of example 40, further comprising:
sending, by the one or more processors, the generated message to the at least one coach identified by the at least one selected coach identifier.

Example 42. The method of example 36, wherein the at least one coach identified by the at least one selected coach identifier includes at least one clinician and/or at least one physician.

Example 43. The method of any one of examples 32 to 42, wherein the performance indicator includes an average number of patients per coach.

Example 44. The method of any one of examples 32 to 43, wherein the number of compliant patients is a number of compliant patients, coached at the at least one selected geographic location.

Example 45. The method of any one of examples 32 to 43, wherein the number of compliant patients is a number of compliant patients, coached by at least one coach identified by the at least one selected coach identifier at the at least one selected geographic location.

Example 46. The method of any one of examples 32 to 45, wherein the at least one selected geographic location includes at least two selected geographic locations, and wherein the performance indicator includes:
a ratio between a number of compliant patients and a total number of patients coached at one of the at least two selected geographic locations; and
a ratio between a number of compliant patients and a total number of patients coached at another one of the at least two selected geographic locations.

Example 47. The method of any one of examples 32 to 46, wherein the plurality of HME devices are provided to the plurality of patients by an HME provider.

Example 48. A method for monitoring performance associated with home medical equipment (HME), comprising:
storing, by a non-transitory computer-readable medium, information for each of a plurality of patients, wherein the information for each patient includes at least one of: (1) an HME provider identifier identifying one of a plurality of HME providers, wherein the one is associated with the patient, (2) a physician identifier identifying a physician who coaches the patient on using a HME device; (3) a geographic location where the patient receives coaching for using the HME device; and (4) an insurer identifier identifying an insurer associated with the patient;
accessing, by one or more processors, device usage data of the plurality of patients that is received from a plurality of HME devices, the HME devices being provided to the patients by a plurality of HME providers;
determining, by the one or more processors, whether each of the plurality of patients is a compliant patient by comparing the device usage data to one or more device usage requirements;
receiving, by the one or more processors, a filter criterion, from a user, the filter criterion including one or more of the following: at least one selected HME provider identifier, at least one selected physician identifier, at least one selected geographic location, and at least one selected insurer identifier; and
generating, by the one or more processors, a performance indicator, for output to the user, based on a number of compliant patients whose information meets the filter criterion.

Example 49. The method of example 48, wherein the plurality of HME providers are of a plurality of patient volumes.

Example 50. The method of any one of examples 48 to 49, wherein the at least one selected HME provider identifier includes at least two selected HME provider identifiers, and wherein the performance indicator includes:
a ratio between a number of compliant patients and a total number of patients associated with one of the at least two selected HME provider identifiers; and
a ratio between a number of compliant patients and a total number of patients associated with another one of the at least two selected HME provider identifiers.

Example 51. The method of any one of examples 48 to 50, wherein the number of compliant patients is a number of compliant patients associated with at least one HME provider identified by the at least one selected HME provider identifier.

Example 52. The method of example 51, wherein the performance indicator indicates a comparison between (1) a ratio between the number of compliant patients and a total number of patients associated with the at least one HME provider identified by the at least one selected HME provider identifier and (2) an average ratio between a number of compliant patients and a total number of patients per HME provider of a patient volume similar to the at least one HME provider identified by the at least one selected HME provider identifier.

Example 53. The method of example 52, further comprising:
generating, by the one or more processors, a message when the ratio associated with the at least one HME provider identified by the at least one selected HME provider identifier falls below the average ratio.

Example 54. The method of example 53, further comprising:
sending, by the one or more processors, the message to the at least one HME provider identified by the at least one selected HME provider identifier.

Example 55. The method of any one of examples 48 to 54, wherein the number of compliant patients is a number of compliant patients associated with at least one HME provider identified by the at least one selected HME provider identifier, and coached at the at least one selected geographic location.

Example 56. The method of example 55, wherein the performance indicator indicates a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider.

Example 57. The method of example 55, wherein the performance indicator indicates a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider of a patient volume similar to the at least one HME provider identified by the at least one selected HME provider identifier.

Example 58. The method of any one of examples 48 to 57, wherein the number of compliant patients is a number of compliant patients, coached by at least one physician identified by the at least one selected physician identifier.

Example 59. The method of any one of examples 48 to 58, wherein the performance indicator includes a ratio between a number of compliant patients and a total number of patients associated with at least one HME provider identified by the at least one HME provider identifier and coached by at least one physician identified by the at least one selected physician identifier.

Example 60. The method of example 59, wherein the performance indicator indicates a comparison between (1) the ratio and (2) an average ratio between a number of compliant patients and a total number of patients coached by at least one physician identified by the at least one selected physician identifier across the plurality of HME providers.

Example 61. The method of any one of examples 48 to 60, wherein the performance indicator includes a ratio between a number of compliant patients and a total number of patients associated with at least one insurer identified by the at least one insurer identifier.

Example 62. The method of example 61, wherein the performance indicator indicates a comparison between (1) the ratio and (2) an average ratio between a number of compliant patients and a total number of patients per insurer.

Example 63. A system for monitoring performance associated with home medical equipment (HME), comprising:
a non-transitory computer-readable medium configured to store information for each of a plurality of patients; and
one or more processors configured to:
access device usage data of the plurality of patients that is repeatedly received from a plurality of HME devices;
determine whether each of the plurality of patients is a compliant patient by comparing the device usage data to one or more device usage requirements;
receive a filter criterion, from a user, the filter criterion concerning the information; and
generate a performance indicator, for output to the user, based on a number of compliant patients whose information meets the filter criterion.

Example 64. The system of example 63, wherein the information for each patient includes at least one of: (1) at least one coach identifier identifying at least one coach who coaches the patient, and (2) a geographic location where the patient receives coaching from the at least one coach.

Example 65. The system of example 64, wherein the filter criterion includes at least one selected coach identifier, at least one selected coach type, at least one therapy device type, and/or at least one selected geographic location.

Example 66. The system of any one of examples 64 to 65, wherein the HME devices are provided to the patients by a plurality of HME providers.

Example 67. The system of example 66, wherein the information for each patient includes at least one of: (1) an HME provider identifier identifying one of the plurality of HME providers associated with the patient, (2) a physician identifier identifying a physician who coaches the patient on using the HME device; (3) a geographic location where the patient receives coaching for using the HME device; (4) an insurer identifier identifying an insurer associated with the patient, and (5) a therapy device type.

Example 68. The system of example 67, wherein the filter criterion includes one or more of the following: at least one selected HME provider identifier, at least one selected physician identifier, at least one selected geographic location, and at least one selected insurer identifier.

Example 69. The system of example 67, wherein the performance indicator indicates an average number of compliant patients coached by an HME provider on a monthly basis.

Example 70. A method for monitoring performance associated with home medical equipment (HME), comprising:
storing, by a non-transitory computer-readable medium, information for each of a plurality of patients;
accessing, by one or more processors, device usage data of the plurality of patient that is repeatedly received from a plurality of HME devices;
determining, by the one or more processors, whether each of the plurality of patients is a compliant patient by comparing the device usage data to one or more device usage requirements;
receiving, by the one or more processors, a filter criterion, from a user, the filter criterion concerning the information; and
generating, by the one or more processors, a performance indicator, for output to the user, based on a number of compliant patients whose information meets the filter criterion.

Example 71. The method of example 70, wherein the information for each patient includes at least one of: (1) at least one coach identifier identifying at least one coach who coaches the patient, and (2) a geographic location where the patient receives coaching from the at least one coach.

Example 72. The method of example 71, wherein the filter criterion includes at least one selected coach identifier, at least one selected coach type, at least one therapy device type, and/or at least one selected geographic location.

Example 73. The method of any one of examples 70 to 72, wherein the HME devices are provided to the patients by a plurality of HME providers.

Example 74. The method of example 73, wherein the information for each patient includes at least one of: (1) an HME provider identifier identifying one of the plurality of HME providers associated with the patient, (2) a physician identifier identifying a physician who coaches the patient on using the HME device; (3) a geographic location where the patient receives coaching for using the HME device; (4) an insurer identifier identifying an insurer associated with the patient, and (5) a therapy device type.

Example 75. The method of example 74, wherein the filter criterion includes one or more of the following: at least one selected HME provider identifier, at least one selected physician identifier, at least one selected geographic location, and at least one selected insurer identifier.

Example 76. The method of any one of examples 70 to 75, wherein the performance indicator indicates an average number of compliant patients coached by an HME provider on a monthly basis.

1. A system for monitoring performance associated with home medical equipment (HME), comprising:
a non-transitory computer-readable medium that stores information for each of a plurality of patients, including, for each patient, one or more of: (a) at least one coach identifier identifying at least one coach who coaches the patient, and (b) a geographic location where the patient receives coaching from the at least one coach;
one or more processors configured to:
access device usage data of the plurality of patients that is repeatedly received from a plurality of HME devices;
determine whether each of the plurality of patients is a compliant patient by comparing the device usage data to one or more device usage requirements;
receive a filter criterion, from a user, the filter criterion including at least one selected coach identifier and/or at least one selected geographic location; and
generate a performance indicator, for output to the user, based on a number of compliant patients whose information meets the filter criterion.

2. The system of claim 1, wherein a device usage data requirement of the one or more device usage requirements indicates a predetermined number of device operation hours.

3. The system of any one of claims 1 to 2, wherein the at least one coach of each patient includes a clinician and/or a physician.

4. The system of any one of claims 1 to 3, wherein the performance indicator includes a ratio between the number of compliant patients and a total number of the plurality of patients.

5. The system of any one of claims 1 to 4, wherein the number of compliant patients is a number of compliant patients, coached by at least one coach identified by the at least one selected coach identifier.

6. The system of claim 5, wherein the performance indicator indicates a progression of the number of compliant patients, coached by the at least one coach identified by the at least one selected coach identifier, over time.

7. The system of claim 5, wherein the performance indicator indicates a comparison between the number of compliant patients, coached by the at least one coach identified by the at least one selected coach identifier and an acceptable threshold.

8. The system of claim 7, wherein the acceptable threshold is an average number of compliant patients per coach.

9. The system of claim 7, wherein the one or more processors is configured to generate a message when the number of compliant patients, coached by the at least one coach identified by the at least one selected coach identifier, falls below the acceptable threshold.

10. The system of claim 9, wherein the one or more processors is configured to send the generated message to the at least one coach identified by the at least one selected coach identifier.

11. The system of claim 5, wherein the at least one coach identified by the at least one selected coach identifier includes at least one clinician and/or at least one physician.

12. The system of any one of claims 1 to 11, wherein the performance indicator includes an average number of patients per coach.

13. The system of any one of claims 1 to 12, wherein the number of compliant patients is a number of compliant patients, coached at the at least one selected geographic location.

14. The system of any one of claims 1 to 12, wherein the number of compliant patients is a number of compliant patients, coached by at least one coach identified by the at least one selected coach identifier at the at least one selected geographic location.

15. The system of any one of claims 1 to 14, wherein the at least one selected geographic location includes at least two selected geographic locations, and wherein the performance indicator includes:
a ratio between a number of compliant patients and a total number of patients coached at one of the at least two selected geographic locations; and
a ratio between a number of compliant patients and a total number of patients coached at another one of the at least two selected geographic locations.

16. The system of any one of claims 1 to 15, wherein the plurality of HME devices are provided to the plurality of patients by an HME provider.

17. A system for monitoring performance associated with home medical equipment (HME), comprising:
a non-transitory computer-readable medium that stores information for each of a plurality of patients, including, for each patient, one or more of: (a) an HME provider identifier identifying one of a plurality of HME providers, wherein the one is associated with the patient, (b) a physician identifier identifying a physician who coaches the patient on using a HME device; (c) a geographic location where the patient receives coaching for using the HME device; and (d) an insurer identifier identifying an insurer associated with the patient; and
one or more processors configured to:
access device usage data of the plurality of patients that is repeatedly received from a plurality of HME devices, the HME devices being provided to the patients by a plurality of HME providers;
determine whether each of the plurality of patients is a compliant patient by comparing the device usage data to one or more device usage requirements;
receive a filter criterion, from a user, the filter criterion including one or more of the following: at least one selected HME provider identifier, at least one selected physician identifier, at least one selected geographic location, and at least one selected insurer identifier; and
generate a performance indicator, for output to the user, based on a number of compliant patients whose information meets the filter criterion.

18. The system of claim 17, wherein each of the plurality of HME providers have a plurality of patients.

19. The system of any one of claims 17 to 18, wherein the at least one selected HME provider identifier includes at least two selected HME provider identifiers, and wherein the performance indicator includes:
a ratio between a number of compliant patients and a total number of patients associated with one of the at least two selected HME provider identifiers; and
a ratio between a number of compliant patients and a total number of patients associated with another one of the at least two selected HME provider identifiers.

20. The system of any one of claims 17 to 19, wherein the number of compliant patients is a number of compliant patients associated with at least one HME provider identified by the at least one selected HME provider identifier.

21. The system of claim 20, wherein the performance indicator indicates a comparison between (1) a ratio between the number of compliant patients and a total number of patients associated with the at least one HME provider identified by the at least one selected HME provider identifier and (2) an average ratio between a number of compliant patients and a total number of patients per HME provider of a patient volume similar to the at least one HME provider identified by the at least one selected HME provider identifier.

22. The system of claim 21, wherein the one or more processors is configured to generate a message when the ratio associated with the at least one HME provider identified by the at least one selected HME provider identifier falls below the average ratio.

23. The system of claim 22, wherein the one or more processors is configured to send the message to the at least one HME provider identified by the at least one selected HME provider identifier.

24. The system of any one of claims 17 to 23, wherein the number of compliant patients is a number of compliant patients associated with at least one HME provider identified by the at least one selected HME provider identifier, and coached at the at least one selected geographic location.

25. The system of claim 24, wherein the performance indicator indicates a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider.

26. The system of claim 24, wherein the performance indicator indicates a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider of a patient volume similar to the at least one HME provider identified by the at least one selected HME provider identifier.

27. The system of any one of claims 17 to 26, wherein the number of compliant patients is a number of compliant patients, coached by at least one physician identified by the at least one selected physician identifier.

28. The system of any one of claims 17 to 27, wherein the performance indicator includes a ratio between a number of compliant patients and a total number of patients associated with at least one HME provider identified by the at least one HME provider identifier and coached by at least one physician identified by the at least one selected physician identifier.

29. The system of claim 28, wherein the performance indicator indicates a comparison between (1) the ratio and (2) an average ratio between a number of compliant patients and a total number of patients coached by at least one physician identified by the at least one selected physician identifier across the plurality of HME providers.

30. The system of any one of claims 17 to 27, wherein the performance indicator includes a ratio between a number of compliant patients and a total number of patients associated with at least one insurer identified by the at least one insurer identifier.

31. The system of claim 30, wherein the performance indicator indicates a comparison between (1) the ratio and (2) an average ratio between a number of compliant patients and a total number of patients per insurer.

32. A method for monitoring performance associated with home medical equipment (HME), comprising:
storing, by a non-transitory computer-readable medium, information for each of a plurality of patients, wherein the information for each patient includes at least one of: (a) at least one coach identifier identifying at least one coach who coaches the patient, and (b) a geographic location where the patient receives coaching from the at least one coach;
accessing, by one or more processors, device usage data of the plurality of patients that is received from a plurality of HME devices;
determining, by the one or more processors, whether each of the plurality of patients is a compliant patient by comparing the device usage data to one or more device usage requirements;
receiving, by the one or more processors, a filter criterion, from a user, the filter criterion including at least one selected coach identifier and/or at least one selected geographic location; and
generating, by the one or more processors, a performance indicator, for output to the user, based on a number of compliant patients whose information meets the filter criterion.

33. The method of claim 32, wherein the device usage requirement indicates a predetermined number of device operation hours.

34. The method of any one of claims 32 to 33, wherein the at least one coach of each patient includes a clinician and/or a physician.

35. The method of any one of claims 32 to 34, wherein the performance indicator includes a ratio between the number of compliant patients and a total number of the plurality of patients.

36. The method of any one of claims 32 to 35, wherein the number of compliant patients is a number of compliant patients, coached by at least one coach identified by the at least one selected coach identifier.

37. The method of claim 36, wherein the performance indicator indicates a progression of the number of compliant patients, coached by the at least one coach identified by the at least one selected coach identifier, over time.

38. The method of claim 36, wherein the performance indicator indicates a comparison between the number of compliant patients, coached by the at least one coach identified by the at least one selected coach identifier and an acceptable threshold.

39. The method of claim 38, wherein the acceptable threshold is an average number of compliant patients per coach.

40. The method of claim 38, further comprising:
generating, by the one or more processors, a message when the number of compliant patients, coached by the at least one coach identified by the at least one selected coach identifier, falls below the acceptable threshold.

41. The method of claim 40, further comprising:
sending, by the one or more processors, the generated message to the at least one coach identified by the at least one selected coach identifier.

42. The method of claim 36, wherein the at least one coach identified by the at least one selected coach identifier includes at least one clinician and/or at least one physician.

43. The method of any one of claims 32 to 42, wherein the performance indicator includes an average number of patients per coach.

44. The method of any one of claims 32 to 43, wherein the number of compliant patients is a number of compliant patients, coached at the at least one selected geographic location.

45. The method of any one of claims 32 to 43, wherein the number of compliant patients is a number of compliant patients, coached by at least one coach identified by the at least one selected coach identifier at the at least one selected geographic location.

46. The method of any one of claims 32 to 45, wherein the at least one selected geographic location includes at least two selected geographic locations, and wherein the performance indicator includes:
a ratio between a number of compliant patients and a total number of patients coached at one of the at least two selected geographic locations; and
a ratio between a number of compliant patients and a total number of patients coached at another one of the at least two selected geographic locations.

47. The method of any one of claims 32 to 46, wherein the plurality of HME devices are provided to the plurality of patients by an HME provider.

48. A method for monitoring performance associated with home medical equipment (HME), comprising:
storing, by a non-transitory computer-readable medium, information for each of a plurality of patients, wherein the information for each patient includes at least one of: (1) an HME provider identifier identifying one of a plurality of HME providers, wherein the one is associated with the patient, (2) a physician identifier identifying a physician who coaches the patient on using a HME device; (3) a geographic location where the patient receives coaching for using the HME device; and (4) an insurer identifier identifying an insurer associated with the patient;
accessing, by one or more processors, device usage data of the plurality of patients that is received from a plurality of HME devices, the HME devices being provided to the patients by a plurality of HME providers;
determining, by the one or more processors, whether each of the plurality of patients is a compliant patient by comparing the device usage data to one or more device usage requirements;
receiving, by the one or more processors, a filter criterion, from a user, the filter criterion including one or more of the following: at least one selected HME provider identifier, at least one selected physician identifier, at least one selected geographic location, and at least one selected insurer identifier; and
generating, by the one or more processors, a performance indicator, for output to the user, based on a number of compliant patients whose information meets the filter criterion.

49. The method of claim 48, wherein the plurality of HME providers are of a plurality of patient volumes.

50. The method of any one of claims 48 to 49, wherein the at least one selected HME provider identifier includes at least two selected HME provider identifiers, and wherein the performance indicator includes:
a ratio between a number of compliant patients and a total number of patients associated with one of the at least two selected HME provider identifiers; and
a ratio between a number of compliant patients and a total number of patients associated with another one of the at least two selected HME provider identifiers.

51. The method of any one of claims 48 to 50, wherein the number of compliant patients is a number of compliant patients associated with at least one HME provider identified by the at least one selected HME provider identifier.

52. The method of claim 51, wherein the performance indicator indicates a comparison between (1) a ratio between the number of compliant patients and a total number of patients associated with the at least one HME provider identified by the at least one selected HME provider identifier and (2) an average ratio between a number of compliant patients and a total number of patients per HME provider of a patient volume similar to the at least one HME provider identified by the at least one selected HME provider identifier.

53. The method of claim 52, further comprising:
generating, by the one or more processors, a message when the ratio associated with the at least one HME provider identified by the at least one selected HME provider identifier falls below the average ratio.

54. The method of claim 53, further comprising:
sending, by the one or more processors, the message to the at least one HME provider identified by the at least one selected HME provider identifier.

55. The method of any one of claims 48 to 54, wherein the number of compliant patients is a number of compliant patients associated with at least one HME provider identified by the at least one selected HME provider identifier, and coached at the at least one selected geographic location.

56. The method of claim 55, wherein the performance indicator indicates a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider.

57. The method of claim 55, wherein the performance indicator indicates a comparison between (1) a ratio between the number of compliant patients and a total number of patients associated with the at least one HME provider identified by the at least one selected HME provider identifier and coached at the at least one selected geographic location and (2) an average ratio between a number of compliant patients and a total number of patients coached at the at least one selected geographic location per HME provider of a patient volume similar to the at least one HME provider identified by the at least one selected HME provider identifier.

58. The method of any one of claims 48 to 57, wherein the number of compliant patients is a number of compliant patients, coached by at least one physician identified by the at least one selected physician identifier.

59. The method of any one of claims 48 to 58, wherein the performance indicator includes a ratio between a number of compliant patients and a total number of patients associated with at least one HME provider identified by the at least one HME provider identifier and coached by at least one physician identified by the at least one selected physician identifier.

60. The method of claim 59, wherein the performance indicator indicates a comparison between (1) the ratio and (2) an average ratio between a number of compliant patients and a total number of patients coached by at least one physician identified by the at least one selected physician identifier across the plurality of HME providers.

61. The method of any one of claims 48 to 60, wherein the performance indicator includes a ratio between a number of compliant patients and a total number of patients associated with at least one insurer identified by the at least one insurer identifier.

62. The method of claim 61, wherein the performance indicator indicates a comparison between (1) the ratio and (2) an average ratio between a number of compliant patients and a total number of patients per insurer.

63. A system for monitoring performance associated with home medical equipment (HME), comprising:
a non-transitory computer-readable medium configured to store information for each of a plurality of patients; and
one or more processors configured to:
access device usage data of the plurality of patients that is repeatedly received from a plurality of HME devices;
determine whether each of the plurality of patients is a compliant patient by comparing the device usage data to one or more device usage requirements;
receive a filter criterion, from a user, the filter criterion concerning the information; and
generate a performance indicator, for output to the user, based on a number of compliant patients whose information meets the filter criterion.

64. The system of claim 63, wherein the information for each patient includes at least one of: (1) at least one coach identifier identifying at least one coach who coaches the patient, and (2) a geographic location where the patient receives coaching from the at least one coach.

65. The system of claim 64, wherein the filter criterion includes at least one selected coach identifier, at least one selected coach type, at least one therapy device type, and/or at least one selected geographic location.

66. The system of any one of claims 64 to 65, wherein the HME devices are provided to the patients by a plurality of HME providers.

67. The system of claim 66, wherein the information for each patient includes at least one of: (1) an HME provider identifier identifying one of the plurality of HME providers associated with the patient, (2) a physician identifier identifying a physician who coaches the patient on using the HME device: (3) a geographic location where the patient receives coaching for using the HME device; (4) an insurer identifier identifying an insurer associated with the patient, and (5) a therapy device type.

68. The system of claim 67, wherein the filter criterion includes one or more of the following: at least one selected HME provider identifier, at least one selected physician identifier, at least one selected geographic location, and at least one selected insurer identifier.

69. The system of claim 67, wherein the performance indicator indicates an average number of compliant patients coached by an HME provider on a monthly basis.

70. A method for monitoring performance associated with home medical equipment (HME), comprising:
storing, by a non-transitory computer-readable medium, information for each of a plurality of patients;
accessing, by one or more processors, device usage data of the plurality of patient that is repeatedly received from a plurality of HME devices;
determining, by the one or more processors, whether each of the plurality of patients is a compliant patient by comparing the device usage data to one or more device usage requirements;
receiving, by the one or more processors, a filter criterion, from a user, the filter criterion concerning the information; and
generating, by the one or more processors, a performance indicator, for output to the user, based on a number of compliant patients whose information meets the filter criterion.

71. The method of claim 70, wherein the information for each patient includes at least one of: (1) at least one coach identifier identifying at least one coach who coaches the patient, and (2) a geographic location where the patient receives coaching from the at least one coach.

72. The method of claim 71, wherein the filter criterion includes at least one selected coach identifier, at least one selected coach type, at least one therapy device type, and/or at least one selected geographic location.

73. The method of any one of claims 70 to 72, wherein the HME devices are provided to the patients by a plurality of HME providers.

74. The method of claim 73, wherein the information for each patient includes at least one of: (1) an HME provider identifier identifying one of the plurality of HME providers associated with the patient, (2) a physician identifier identifying a physician who coaches the patient on using the HME device; (3) a geographic location where the patient receives coaching for using the HME device; (4) an insurer identifier identifying an insurer associated with the patient, and (5) a therapy device type.

75. The method of claim 74, wherein the filter criterion includes one or more of the following: at least one selected HME provider identifier, at least one selected physician identifier, at least one selected geographic location, and at least one selected insurer identifier.

76. The method of any one of claims 70 to 75, wherein the performance indicator indicates an average number of compliant patients coached by an HME provider on a monthly basis.
